SEQUENCE LISTING (72 sequences)

SEQ ID NO: 1 - DNA, 155 bp, Artificial Sequence
  Synthetic - Primer
gcgctaatacgactcactatagggaattacaatacaaatagaggagacggacaatgtacaccatcgtcgagcaatatgaaagagctgaggctcggcactcaaccaacctggcggcagcgcaaaaggagacaggtacgttaatagttaatagcgta

SEQ ID NO: 2 - DNA, 126 bp, Artificial Sequence
  Synthetic - Primer
ttatcagccacctcctgtaatcccagcggcattgacatactcgtgcagaaccatgtgatcgcgcgatccaccgccactacctccgccgtcactcaggttaacaatattgcagcagtacgcacacaa

SEQ ID NO: 3 - DNA, 155 bp, Artificial Sequence
  Synthetic - Primer
gcgctaatacgactcactatagggaattacaatacaaatagaggagacggacaatgtacaccatcgtcgagcaatatgaaagagctgaggctcggcactcaaccaacctggcggcagcgcaaaagcaggaactaatcagacaaggaactgattac

SEQ ID NO: 4 - DNA, 126 bp, Artificial Sequence
  Synthetic - Primer
ttatcagccacctcctgtaatcccagcggcattgacatactcgtgcagaaccatgtgatcgcgcgatccaccgccactacctccgccgtcactcagcaaccacgttcccgaaggtgtgacttccat

SEQ ID NO: 5 - DNA, 155 bp, Artificial Sequence
  Synthetic - Primer
gcgctaatacgactcactatagggaattacaatacaaatagaggagacggacaatgtacaccatcgtcgagcaatatgaaagagctgaggctcggcactcaaccaacctggcggcagcgcaaaagtctgataatggaccccaaaatcagcgaaat

SEQ ID NO: 6 - DNA, 126 bp, Artificial Sequence
  Synthetic - Primer
ttatcagccacctcctgtaatcccagcggcattgacatactcgtgcagaaccatgtgatcgcgcgatccaccgccactacctccgccgtcactcagtccattctggttactgccagttgaatctga

SEQ ID NO: 7 - DNA, 155 bp, Artificial Sequence
  Synthetic - Primer
gcgctaatacgactcactatagggaattacaatacaaatagaggagacggacaatgtacaccatcgtcgagcaatatgaaagagctgaggctcggcactcaaccaacctggcggcagcgcaaaaggttcaacaatggggttttacaggtaaccta

SEQ ID NO: 8 - DNA, 126 bp, Artificial Sequence
  Synthetic - Primer
ttatcagccacctcctgtaatcccagcggcattgacatactcgtgcagaaccatgtgatcgcgcgatccaccgccactacctccgccgtcactcagagtccagtcaacacgcttaacaaagcactc

SEQ ID NO: 9 - DNA, 156 bp, Artificial Sequence
  Synthetic - Primer
tttctaatacgactcactatagggatcagcagcataaacagaggagaccagacatgtataccatcgttgaacaatacgaacgcgcagaagcaagacactccaccaacctcgccgccgcccaaaaattgttggcctttaccagacattttgctctca

SEQ ID NO: 10 - DNA, 130 bp, Artificial Sequence
  Synthetic - Primer
ttattatcctcctcccgttattcccgccgcgtttacgtattcatgcagtaccatatgatccctcgagccacctcctgatcctcctccgtcgcttaaataggcagcagtaggggaacttctcctgctagaa

SEQ ID NO: 11 - DNA, 159 bp, Artificial Sequence
  Synthetic - Primer
caactaatacgactcactatagggataaacagaagcaacagaggagaccaaggatgtatacaatcgtcgaacaatatgaaagagccgaagcccgccacagcacgaacttagccgccgcccaaaaaagcattgttagcaggattgcgggtgccaatgtga

SEQ ID NO: 12 - DNA, 129 bp, Artificial Sequence
  Synthetic - Primer
ttattagccaccgccggttatgcccgctgcatttacatattcgtgcagcaccatgtgatctcgggacccgccgcctgaacctcctccatcacttaagaagacggcatcatatgggttgcaactgaggga

SEQ ID NO: 13 - DNA, 156 bp, Artificial Sequence
  Synthetic - Primer
aaactaatacgactcactatagggacaagaacaataagcagaggagatggacaatgtatactatcgtcgaacaatacgaacgcgccgaagcacgccattccaccaacttagcggcggcacagaaacttgttagcaggattgcgggtgccaatgtga

SEQ ID NO: 14 - DNA, 132 bp, Artificial Sequence
  Synthetic - Primer
ttattatccgccgccggtgatgcccgccgcgttaacgtattcgtgtagtaccatgtgatctctgcttccgcctcccgagcctccgccgtcagataaaacaaagacggcatcatatgggttgcaactgaggga

SEQ ID NO: 15 - DNA, 157 bp, Artificial Sequence
  Synthetic - Primer
gaactaatacgactcactatagggcacagaaacaggaacagaggagatagaacatgtatacgatcgtcgaacaatatgaaagagcagaagcaagacacagcacaaacctagcagcggcccagaaaaagcagtattattgggtaaaccttggggccga

SEQ ID NO: 16 - DNA, 133 bp, Artificial Sequence
  Synthetic - Primer
ttattaacctccacccgtaattcccgctgcgtttacatattcatgcagcaccatatgatccctgctgcctcctccgcttccgcctccgtcgcttagtgtttggtggaccctcagattcaactggcagtaacca

SEQ ID NO: 17 - DNA, 186 bp, Artificial Sequence
  Synthetic - Primer
ttatcagccacctccctcttcgctattacgccagctggcgaaagggggatgtgctgcaaggcgattaagttgggtaacgccagggttttcccagtcacgacgttgaagcactacagcaagtgaatccgtaatcatggaaccgcccccgtcactcagcaaccacgttcccgaaggtgtgacttccat

SEQ ID NO: 18 - DNA, 189 bp, Artificial Sequence
  Synthetic - Primer
ttatcaccccccaccttcctcactgttacgccaggaggcaaatggtggatgagccgccagacggtttaattgagtaacacctggattttcccaatcacggcgctgaagcactacagcaagtgaatccgtaatcatggaaccgcccccggagtcggacagcaaccacgttcccgaaggtgtgacttccat

SEQ ID NO: 19 - DNA, 86 bp, Artificial Sequence
  Synthetic - Primer
gcgctaatacgactcactatagggaattacaatacaaatagaggagacggacaatgttccatgccaatgcgcgacattccgaagaa

SEQ ID NO: 20 - DNA, 127 bp, Artificial Sequence
  Synthetic - Primer
ttatcagccacctcctgtaatcccagcggcattgacatactcgtgcagaaccatgtgatcgcgcgatccaccgccactacctccgccgtcactcagcaaggaactgattacaaacattggccgcaaa

SEQ ID NO: 21 - DNA, 89 bp, Artificial Sequence
  Synthetic - Primer
gcgctaatacgactcactatagggaattacaatacaaatagaggagacggacaatggttgaagaagtacaaaatgtcattaatgctatg

SEQ ID NO: 22 - DNA, 171 bp, Artificial Sequence
  Synthetic - Primer
ttaggttgagtgccgagcctcagctctttcatattgctcgacgatggtgtagcccccggatcccccggttgagtgccgagcctcagctctttcatattgctcgacgatggtgtacgatccaccgccactacctccgccaagtttcagcatgcaaaatttgcaaaatatgtg

SEQ ID NO: 23 - DNA, 89 bp, Artificial Sequence
  Synthetic - Primer
gcgctaatacgactcactatagggaattacaatacaaatagaggagacggacaatgtttgaaaaccccaagggactaattcatggttgt

SEQ ID NO: 24 - DNA, 171 bp, Artificial Sequence
  Synthetic - Primer
ttaggttgagtgccgagcctcagctctttcatattgctcgacgatggtgtagcccccggatcccccggttgagtgccgagcctcagctctttcatattgctcgacgatggtgtacgatccaccgccactacctccgcccatttctatgcttgtttcccgactgtggttaac

SEQ ID NO: 25 - DNA, 89 bp, Artificial Sequence
  Synthetic - Primer
gcgctaatacgactcactatagggaattacaatacaaatagaggagacggacaatgtcatttgttaaaactaaatgtaagaaaaatctg

SEQ ID NO: 26 - DNA, 171 bp, Artificial Sequence
  Synthetic - Primer
ttaggttgagtgccgagcctcagctctttcatattgctcgacgatggtgtagcccccggatcccccggttgagtgccgagcctcagctctttcatattgctcgacgatggtgtacgatccaccgccactacctccgccttctctaatgttattacggctaattgtgctcac

SEQ ID NO: 27 - DNA, 89 bp, Artificial Sequence
  Synthetic - Primer
gcgctaatacgactcactatagggaattacaatacaaatagaggagacggacaatgacatcaaaaagtatctttttgaaagttaaagta

SEQ ID NO: 28 - DNA, 171 bp, Artificial Sequence
  Synthetic - Primer
ttaggttgagtgccgagcctcagctctttcatattgctcgacgatggtgtagcccccggatcccccggttgagtgccgagcctcagctctttcatattgctcgacgatggtgtacgatccaccgccactacctccgccgactgaataaggggactgatttgtgtaacaagt

SEQ ID NO: 29 - DNA, 88 bp, Artificial Sequence
  Synthetic - Primer
tttctaatacgactcactatagggaattacaatacaaatagaggagacggacaatgagactactgaacactgaaggagaaatccagat

SEQ ID NO: 30 - DNA, 83 bp, Artificial Sequence
  Synthetic - Primer
tcacgtgatgccagcggcattcacatactcgtgcagtaccatgtggtcacgagtccttttgctcacctgtggtatcactccaa

SEQ ID NO: 31 - DNA, 89 bp, Artificial Sequence
  Synthetic - Primer
tttctaatacgactcactatagggaattacaatacaaatagaggagacggacaatgatccagatcgatggtgtgtcttgggattcaata

SEQ ID NO: 32 - DNA, 83 bp, Artificial Sequence; identical to SEQ ID NO: 30
  Synthetic - Primer

SEQ ID NO: 33 - DNA, 87 bp, Artificial Sequence
  Synthetic - Primer
tttctaatacgactcactatagggaattacaatacaaatagaggagacggacaatggctttccttttctcacttttcctaccaatgc

SEQ ID NO: 34 - DNA, 84 bp, Artificial Sequence
  Synthetic - Primer
tcacgtgatgccagcggcattcacatactcgtgcagtaccatgtggtcacgtggattcacgcttgatgacttctttatttctct

SEQ ID NO: 35 - DNA, 83 bp, Artificial Sequence
  Synthetic - Primer
tttctaatacgactcactatagggaattacaatacaaatagaggagacggacaatgcagcatgattttgaagtcagaggagat

SEQ ID NO: 36 - DNA, 81 bp, Artificial Sequence
  Synthetic - Primer
tcacgtgatgccagcggcattcacatactcgtgcagtaccatgtggtcacggatttttgtcaacttgagggagggagcttt

SEQ ID NO: 37 - DNA, 86 bp, Artificial Sequence
  Synthetic - Primer
tttctaatacgactcactatagggaattacaatacaaatagaggagacggacaatgatgctgatcccctgtgtgagagaaaagaat

SEQ ID NO: 38 - DNA, 83 bp, Artificial Sequence
  Synthetic - Primer
tcacgtgatgccagcggcattcacatactcgtgcagtaccatgtggtcacgctccccatcatgtgagtcatcagaacctaaca

SEQ ID NO: 39 - DNA, 89 bp, Artificial Sequence
  Synthetic - Primer
tttctaatacgactcactatagggaattacaatacaaatagaggagacggacaatgattgttagcaatttcaacagtctaatcaatgtc

SEQ ID NO: 40 - DNA, 82 bp, Artificial Sequence
  Synthetic - Primer
tcacgtgatgccagcggcattcacatactcgtgcagtaccatgtggtcacgttttatcttacagtcagaaatgaagaagcat

SEQ ID NO: 41 - DNA, 88 bp, Artificial Sequence
  Synthetic - Primer
tttctaatacgactcactatagggaattacaatacaaatagaggagacggacaatggaacagaaagaaatggatttatctgctcttcg

SEQ ID NO: 42 - DNA, 83 bp, Artificial Sequence
  Synthetic - Primer
tcacgtgatgccagcggcattcacatactcgtgcagtaccatgtggtcacgatcataggaatcccaaattaatacactcttgt

SEQ ID NO: 43 - DNA, 134 bp, Artificial Sequence
  Synthetic - Primer
tttctaatacgactcactatagggaattacaatacaaatagaggagacggacaatgtacaccatcgtcgagcaatatgaaagagctgaggctcggcactcaacctctttagctacaccacccacccttagttct

SEQ ID NO: 44 - DNA, 81 bp, Artificial Sequence
  Synthetic - Primer
tcacgtgatgccagcggcattcacatactcgtgcagtaccatgtggtcacgtctatcatttttcttcagactttcatcatg

SEQ ID NO: 45 - DNA, 137 bp, Artificial Sequence
  Synthetic - Primer
tttctaatacgactcactatagggaattacaatacaaatagaggagacggacaatgtacaccatcgtcgagcaatatgaaagagctgaggctcggcactcaaccgttgaagaagtacaaaatgtcattaatgctatg

SEQ ID NO: 46 - DNA, 84 bp, Artificial Sequence
  Synthetic - Primer
tcacgtgatgccagcggcattcacatactcgtgcagtaccatgtggtcacgaagtttcagcatgcaaaatttgcaaaatatgtg

SEQ ID NO: 47 - DNA, 134 bp, Artificial Sequence
  Synthetic - Primer
tttctaatacgactcactatagggaattacaatacaaatagaggagacggacaatgtacaccatcgtcgagcaatatgaaagagctgaggctcggcactcaaccaatgagcatgattttgaagtcagaggagat

SEQ ID NO: 48 - DNA, 81 bp, Artificial Sequence
  Synthetic - Primer
tcacgtgatgccagcggcattcacatactcgtgcagtaccatgtggtcacgatagcaacagatttctagccccctgaagat

SEQ ID NO: 49 - DNA, 134 bp, Artificial Sequence
  Synthetic - Primer
tttctaatacgactcactatagggaattacaatacaaatagaggagacggacaatgtacaccatcgtcgagcaatatgaaagagctgaggctcggcactcaaccgtagatctgaatgctgatcccctgtgtgag

SEQ ID NO: 50 - DNA, 81 bp, Artificial Sequence
  Synthetic - Primer
tcacgtgatgccagcggcattcacatactcgtgcagtaccatgtggtcacgctccccatcatgtgagtcatcagaacctaa

SEQ ID NO: 51 - DNA, 137 bp, Artificial Sequence
  Synthetic - Primer
tttctaatacgactcactatagggaattacaatacaaatagaggagacggacaatgtacaccatcgtcgagcaatatgaaagagctgaggctcggcactcaacctttgaaaaccccaagggactaattcatggttgt

SEQ ID NO: 52 - DNA, 84 bp, Artificial Sequence
  Synthetic - Primer
tcacgtgatgccagcggcattcacatactcgtgcagtaccatgtggtcacgcatttctatgcttgtttcccgactgtggttaac

SEQ ID NO: 53 - DNA, 137 bp, Artificial Sequence
  Synthetic - Primer
tttctaatacgactcactatagggaattacaatacaaatagaggagacggacaatgtacaccatcgtcgagcaatatgaaagagctgaggctcggcactcaaccacatcaaaaagtatctttttgaaagttaaagta

SEQ ID NO: 54 - DNA, 84 bp, Artificial Sequence
  Synthetic - Primer
tcacgtgatgccagcggcattcacatactcgtgcagtaccatgtggtcacggactgaataaggggactgatttgtgtaacaagt

SEQ ID NO: 55 - DNA, 134 bp, Artificial Sequence
  Synthetic - Primer
tttctaatacgactcactatagggaattacaatacaaatagaggagacggacaatgtacaccatcgtcgagcaatatgaaagagctgaggctcggcactcaacctgtgcagcatttgaaaaccccaagggacta

SEQ ID NO: 56 - DNA, 84 bp, Artificial Sequence; identical to SEQ ID NO: 52
  Synthetic - Primer

SEQ ID NO: 57 - DNA, 134 bp, Artificial Sequence
  Synthetic - Primer
tttctaatacgactcactatagggaattacaatacaaatagaggagacggacaatgtacaccatcgtcgagcaatatgaaagagctgaggctcggcactcaaccaggaaaactttgaggaacattcaatgtcac

SEQ ID NO: 58 - DNA, 86 bp, Artificial Sequence
  Synthetic - Primer
tcacgtgatgccagcggcattcacatactcgtgcagtaccatgtggtcacgaacattttctctaatgttattacggctaattgtgc

SEQ ID NO: 59 - DNA, 135 bp, Artificial Sequence
  Synthetic - Primer
tttctaatacgactcactatagggaattacaatacaaatagaggagacggacaatgtacaccatcgtcgagcaatatgaaagagctgaggctcggcactcaaccttttcaaatccaggaaatgcagaagaggaat

SEQ ID NO: 60 - DNA, 82 bp, Artificial Sequence
  Synthetic - Primer
tcacgtgatgccagcggcattcacatactcgtgcagtaccatgtggtcacgctgtacaggcttgatattagactcattcttt

SEQ ID NO: 61 - DNA, 27 bp, Artificial Sequence
  Synthetic - Exemplary pathogen in-frame sequence shown in FIG. 2
aaaccaccaaggtccaaagcgagcaag

SEQ ID NO: 62 - protein, 9 aa, Artificial Sequence
  Synthetic - Exemplary pathogen in-frame sequence shown in FIG. 2
KPPRSKASK

SEQ ID NO: 63 - DNA, 27 bp, Artificial Sequence
  Synthetic - Exemplary wild-type sequence shown in FIG. 4
aaaccaccaaggtccatagctagcaag

SEQ ID NO: 64 - protein, 9 aa, Artificial Sequence
  Synthetic - Exemplary wild-type sequence shown in FIG. 4
KPPRSIASK

SEQ ID NO: 65 - DNA, 26 bp, Artificial Sequence
  Synthetic - Exemplary deletion mutant sequence shown in FIG. 4
aaaccaccaaggtcatagctagcaag

SEQ ID NO: 66 - protein, 5 aa, Artificial Sequence
  Synthetic - Exemplary deletion mutant sequence shown in FIG. 4
KPPRS

SEQ ID NO: 67 - DNA, 28 bp, Artificial Sequence
  Synthetic - Exemplary insertion mutant sequence shown in FIG. 4
aaaccaccaaggtcgcatagctagcaag

SEQ ID NO: 68 - protein, 7 aa, Artificial Sequence
  Synthetic - Exemplary insertion mutant sequence shown in FIG. 4
KPPRSHS

SEQ ID NO: 69 - DNA, 27 bp, Artificial Sequence
  Synthetic - Exemplary nonsense mutant sequence shown in FIG. 4
aaaccaccaaggtccatagctagctag

SEQ ID NO: 70 - protein, 8 aa, Artificial Sequence
  Synthetic - Exemplary nonsense mutant sequence shown in FIG. 4
KPPRSIAS

SEQ ID NO: 71 - DNA, 30 bp, Artificial Sequence
  Synthetic - Primer
acgaacaaactaaaatgtctgataatggac

SEQ ID NO: 72 - DNA, 30 bp, Artificial Sequence
  Synthetic - Primer
gttttgatcgcgccccactgcgttctccat

(19) United States
(12) Patent Application Publication
(10) Pub. No.: US 2023/0366040 A1
(43) Pub. Date: Nov. 16, 2023

(54) RAPID AND LOW-COST NUCLEIC ACID DETECTION USING TRANSLATION-BASED SEQUENCE VERIFICATION ASSAYS

(71) Applicant: ARIZONA BOARD OF REGENTS ON BEHALF OF ARIZONA STATE UNIVERSITY, Scottsdale, AZ (US)

(72) Inventors: Alexander GREEN, Boston, MA (US); Kaiyue WU, Tempe, AZ (US)

(21) Appl. No.: 18/021,719

(22) PCT Filed: Aug. 26, 2021
(86) PCT No.: PCT/US2021/047676

§ 371 Date: Feb. 16, 2023

Related U.S. Application Data
(60) Provisional application No. 63/070,537, filed on Aug. 26, 2020.

(51) Int. Cl. C12Q 1/70; C12Q 1/6886; C12Q 1/6897

(52) U.S. Cl. CPC C12Q 1/701; C12Q 1/6886; C12Q 1/6897; C12Q 2600/156; C12Q 1/6804

(57) ABSTRACT

Provided herein are methods and compositions for rapid, highly sensitive detection of a target nucleic acid in a sample, which may be indicative of the presence of a pathogen, disease condition, or disease predisposition. In particular, provided herein is a low-cost, portable method for detecting target nucleic acid sequences that rapidly provides reliable, visible test results and does not require elaborate biosafety precautions or sophisticated laboratory equipment.

### CROSS-REFERENCE TO RELATED APPLICATIONS

This application claims the benefit of, and priority to, U.S. Provisional Patent Application 63/070,537 filed Aug. 26, 2020, the contents of which are hereby incorporated by reference in their entirety.

### STATEMENT REGARDING FEDERALLY FUNDED RESEARCH OR DEVELOPMENT

This invention was made with government support under R21 AI136571 awarded by the National Institutes of Health and 2029532 awarded by the National Science Foundation. The government has certain rights in the invention.

### SEQUENCE LISTING

A Sequence Listing accompanies this application and is submitted as an ASCII text file of the sequence listing named “112624_01283_ST25.txt” which is 21,829 bytes in size and was created on Aug. 25, 2021. The sequence listing is electronically submitted via EFS-Web with the application and is incorporated herein by reference in its entirety.

### BACKGROUND

Rapid, low-cost, instrument-free diagnostics for nucleic acid detection are important tools for identifying diseases, monitoring health conditions, directing treatment, and managing infectious diseases. For example, amid the COVID-19 outbreak, detection of SARS-CoV-2 viral RNA has proved to be essential for identifying the virus and combatting its spread. Rapid instrument-free tests can lead to early detection, which ensures timely treatment for patients and appropriate mitigation strategies to prevent disease spread. In addition, nucleic acid detection provides useful information for hereditary or genetic diseases. For example, patients with mutations in the BRCA1 and BRCA2 genes have increased likelihood to develop breast and ovarian cancer. The ability to detect these variants can inform patients of the risks of certain diseases and select proper treatments and courses of action.

Currently, PCR-based diagnostics are the main strategies for detecting nucleic acids in clinical practices; however, they require expensive instruments and trained technicians and therefore PCR-based diagnostics are usually limited to centralized laboratories. These infrastructure requirements substantially increase both the cost and time required to return assay results. Accordingly, there remains a need in the art for rapid and low-cost nucleic acid detection platforms for point-of-care genetic testing and virus detection.

### SUMMARY OF THE DISCLOSURE

This disclosure is related to methods and compositions for rapid, low-cost nucleic acid detection that employ cell-free transcription-translation reactions along with peptide reporters to provide convenient portable assays.

In a first aspect, provided herein is a method of detecting a target nucleic acid in a sample. The method can comprise or consist essentially of the steps of: (a) amplifying a nucleic acid in a sample using a forward peptide primer and a reverse peptide primer, wherein the forward peptide primer comprises, from 5′ to 3′, (1) a nucleic acid sequence encoding a ribosome binding site (RBS), (2) a start codon, and (3) a nucleic acid sequence of a first portion of a target nucleic acid sequence, wherein the first portion is optionally a protein coding sequence in-frame with the start codon; and wherein the reverse peptide primer comprises, from 3′ to 5′, (1) a nucleic acid sequence complementary to a second portion of the target nucleic acid sequence, (2) a reverse complement of a sequence encoding a peptide reporter, and (3) a reverse complement of a stop codon in-frame with the reverse complement of the sequence encoding the peptide reporter; wherein the amplified product comprises the target nucleic acid sequence which includes a protein coding sequence (i) between the first portion and the second portion and (ii) in-frame with a sequence encoding the peptide reporter; (b) translating the amplified nucleic acid into translation products using a cell-free translation reaction; and (c) detecting the translated peptide reporter among the translation products wherein the presence of the peptide reporter indicates the presence of the target nucleic acid in the sample.

The target nucleic acid can comprise a frame-altering mutation or a nonsense mutation, and the forward peptide primer can further comprise, 3′ to the start codon and 5′ to the first portion of the target nucleic acid sequence, a nucleic acid sequence encoding a second peptide reporter in-frame with the start codon and in-frame with the first portion of the target nucleic acid sequence. Then, the detecting step (c) may comprise detecting at least one of the translated first and second peptide reporters among the translation products wherein the presence of the first peptide reporter indicates there is not a frame shift or nonsense mutation in the target nucleic acid. If the translated first peptide reporter is not detected, but the translated first peptide reporter is not detected, then the target nucleic acid is present in the sample, but there may be a mutation, such as a frame shift or nonsense mutation.

The target nucleic acid can comprise a single nucleotide polymorphism (SNP) or single nucleotide variant (SNV), and the forward primer can comprise one or more nucleotides corresponding to a SNP or SNV of interest within the nucleic acid sequence of the first portion of the target nucleic acid sequence. The detecting step (c) can indicate the presence in the sample of a target nucleic acid comprising a SNP or SNV of interest.

In another aspect, provided herein is a composition comprising: (a) a forward primer comprising, from 5′ to 3′, a nucleic acid sequence encoding a ribosome binding site (RBS), a start codon, and a nucleic acid sequence of a first portion of a target nucleic acid sequence; and/or (b) a reverse primer comprising, from 5′ to 3′, a reverse complement of a stop codon in-frame with the reverse complement of the sequence encoding a first peptide reporter in frame with a nucleic acid sequence complementary to a second portion of the target nucleic acid sequence. In some embodiments, the forward primer comprises from 5′ to 3′: (a) a transcriptional promoter, a nucleic acid sequence encoding a ribosome binding site (RBS), a start codon, and a nucleic acid sequence of a first portion of a target nucleic acid sequence; (b) a nucleic acid sequence encoding a ribosome binding site (RBS), a start codon, a nucleic acid sequence encoding a second peptide reporter in-frame with the start codon, and a nucleic acid sequence of a first portion of a target nucleic acid sequence; or (c) a transcriptional promoter, a nucleic acid sequence encoding a ribosome binding site (RBS), a start codon, a nucleic acid sequence encoding a second peptide reporter in-frame with the start codon, and a nucleic acid sequence of a first portion of a target nucleic acid sequence.

Provided herein is a composition comprising a forward primer and a reverse primer, wherein the respective forward and reverse primers comprises a nucleotide sequence having at least 90%, 92%, 94%, 95%, 96%, 97%, 98%, 99%, or 100% identity respectively to SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, and SEQ ID NO: 59 and SEQ ID NO: 60.

In a further aspect, provided herein is a kit comprising: (a) a forward and reverse primer composition described herein, and (b) a reagent or device.

### BRIEF DESCRIPTION OF THE DRAWINGS

The patent or patent application file contains at least one drawing in color. Copies of this patent or patent application publication with color drawings will be provided by the Office upon request and payment of the necessary fee.

FIG. 1 provides a schematic overview of the translational-based nucleic acid detection platform using peptide reporters. (A) Reporting primer design strategies and expected amplified DNA sequence. The 5′ signal region of the forward primer may contain a T7 promoter, a ribosome binding site and the start codon sequence. The 5′ signal region of the reverse primer may contain a sequence encoding the peptide reporter and one or more stop codon sequences in-frame with the sequence encoding the peptide reporter (* indicates complementary sequence). These primers append transcription-translation signal elements and a peptide reporter encoding sequence to the target sequence following amplification. (B) Applications of the platform in detecting the presence of nucleic acids and mutations. Confirmation of the presence or sequence of an amplification product can be achieved by altering primer specificity to only allow binding and amplification of targeted sequence, which produces signal output. Detection of frame-shift and nonsense mutations can be achieved by translation of the products, where frame-shifted and early terminated products will not produce downstream reporter peptides. (C) Example of scheme used for applying the reporting system for nucleic acid detection. Primer design is completed in silico. Reporter primers are mixed with extracted nucleic acids in an amplification reaction. Amplified product is then added to a cell-free transcription-translation reaction in a test tube or on a paper substrate. The transcription-translation products will provide fluorescence or color output based on the reporter peptide used.

FIG. 2 demonstrates detection of pathogen nucleic acids and SNV mutations. (A) Schematic representation of the amplified DNA configuration and synthesized peptide with GFP11 reporter. The amplified DNA product containing an open reading frame is able to express the reporter peptide to produce the output fluorescence signal. (B) The self-assembling mechanism of split GFP. The short GFP11 peptide self-assembles with GFP1-10 to produce fluorescence. (C) The detection pipeline for pathogens. Samples with the target pathogen can be amplified by the primers to produce signal output while samples without pathogens cannot. (D-G) Time-course measurement of green fluorescence for SARS-CoV-2 targeting the E gene (D), N gene (E), N gene (F), and ORF1b (G). Primers targeting the E and N genes with GFP11 reporters were used to detect synthetic viral RNA. RNA (at a concentration of ˜40 nM) was amplified using RT-RPA at 37 ° C. for 30 minutes. RT-RPA products were added to cell-free reactions, and fluorescence was measured. Graphed on the x-axis is time in minutes and on the y-axis is fluorescence in arbitrary units (a.u.).

FIG. 3 demonstrates detection of single nucleotide polymorphisms (SNPs) (or related single nucleotide variant (SNV) mutations). (A) The detection pipeline for SNP. Wild-type samples can be amplified by the primers to produce signal output while SNP samples cannot. (B and C) Time-course measurement of green fluorescence for the H63D mutant related to hemochromatosis (B) and the T215F (C) mutation which leads to HIV drug resistance. WT and mutant DNA were amplified by RPA at 37° C. for 30 minutes. RPA products were added to cell-free reactions, and fluorescence was measured. Graphed on the x-axis is time in minutes and on the y-axis is fluorescence in arbitrary units (a.u.).

FIG. 4 demonstrates detection of frame-alternating genetic mutations. (A) The pipeline for detecting frame-alternating mutations. Both wild-type and mutant samples can be amplified by primers, but only samples having a valid open reading frame (an open reading frame extending through the reverse primer's target nucleic acid homology sequence) will result in the expression of the reporter peptide. (B) Examples of frame-altering mutations causing early termination of ORFs. Downstream GFP11 peptide sequences are designed to be in-frame with the wild-type sequence, thus allowing subsequent expression of GFP11 which provides fluorescence. (C) Time-course measurement of green fluorescence detecting BRCA-2679_2682delGAAA. WT and mutant synthetic DNA samples were amplified by PCR. The PCR products are added to the cell-free reaction and green fluorescence signal was measured. (D) Time-course measurement of green fluorescence detecting BRCA1_5266dupC. WT and mutant synthetic DNA samples were amplified by PCR. The PCR products are added to the cell-free reaction and green fluorescence signal was measured. (E,F) Time-course measurement of green fluorescence for WT and the frameshift mutants BRCA2-6147delC (E) and BRCA1-5182insC (F). In (C-F), the x-axis is time in minutes, and the y-axis is fluorescence in arbitrary units (a.u.). In (C-D, F), the fluorescence is reported in a.u.×10⁴, and, in (E), the fluorescence is reported in a.u.×10⁵. (G) The bar plot of fluorescence (a.u.) of green fluorescence for WT and mutant samples of 8 pathogenic mutations in the BRCA1 and BRCA2 genes. (H) Photograph of green fluorescence observed from cell-free reactions under blue light illumination with an orange optical filter.

FIG. 5 demonstrates dual fluorescence output for assay self-calibration. (A) Primer design strategy for a self-calibrating detection platform against frame-alternating mutations. sfCherry11 sequence was inserted into the forward primers permitting production of calibrating sfCherry11 signal even if the downstream reading frame contains a stop codon. (B) The detection pipeline for a self-calibrating platform using two different split fluorescence proteins. Both wild-type and mutant samples can be amplified by primers and produce red fluorescence, but only samples having wild-type open reading frames will express the GFP11 reporter peptide and produce green fluorescence. (C) Time-course measurement of green fluorescence detecting BRCA-2679_2682delGAAA using the self-calibrating system. WT and mutant synthetic DNA samples were amplified by PCR. The PCR products were added to the cell-free reaction, and green fluorescence signal was measured. (D) Time-course measurement of red fluorescence detecting BRCA-2679_2682delGAAA using the self-calibrating system. WT and mutant synthetic DNA samples were amplified by PCR. The PCR products were added to the cell-free reaction, and red fluorescence signal was measured. In (C-D), the x-axis is time in minutes, and the y-axis is fluorescence in arbitrary units (a.u.).

FIG. 6 demonstrates dual fluorescence output for assay self-calibration for viral strain identification assays. (A) The detection pipeline for a self-calibrating platform using two split fluorescence proteins in virus identification. Both the target virus and a closely related virus can be amplified by primers and produce red fluorescence, but only samples having open reading frame from the correct virus will express the GFP11 reporter peptide and produce green fluorescence. (B, C, D, E) Discrimination of SARS-CoV-2 from SARS-CoV using the self-calibrating system from mutations in different regions and frames of the antisense orientation of the SARS-CoV-2 N gene. The x-axis is time in minutes, and the y-axis is fluorescence in arbitrary units (a.u.).

FIG. 7 demonstrates integration of recombinase polymerase amplification for detection nucleic acids from serum samples and paper-based colorimetric readouts using a lacZ-α peptide reporter. (A) Schematic of isothermal recombinase polymerase amplification (RPA). The recombinase first binds to the primers and directs them to complementary regions on the target sequences. Single-stranded binding protein binds to stabilize the structure, allowing the DNA polymerase to initiate amplification. (B and C) Detection of the frameshift mutations in serum samples with green (B) and red (C) fluorescence readouts. BRCA1-3029_3030del is present on one allele, another allele remains wild-type, which leads to an elevated green fluorescence signal in the mutant samples. The x-axis is time in minutes, and the y-axis is fluorescence in arbitrary units (a.u.). In (B), the fluorescence is reported in a.u.×10⁴. (D, E) The limit of detection of the reporting platform. In (D), the fluorescence in arbitrary units (a.u.) is shown for different concentrations of nucleic acid: 800 aM, 80 aM, and 8 aM. The detection limit of the platform is measured to be between 8 attomolar (aM) and ˜80 aM when RPA is used. In (E), graphed on the x-axis is time in minutes and on the y-axis is fluorescence in arbitrary units (a.u.). (F) Schematic of lacZ α-complementation and the color change reaction on paper discs catalyzed by assembled lacZ enzyme. (G, H) Paper-based colorimetric reaction of BRCA2 c.658_659del BRCA2 c.4936_4939del using lacZ-α as reporter. The images were taken after 2 hours of cell-free reaction. The yellow substrate will turn purple after reaction with the assembled lacZ enzyme.

FIG. 8 demonstrates detection of SARS-CoV-2 using the peptide primers down to a detection limit of 50 aM using a GFP11 peptide reporter coupled with GFP1-10. Viral RNA was amplified using consecutive RT-RPA and RPA reactions with different primers and then the amplification products applied to a cell-free reaction. On the x-axis is time in minutes and on the y-axis is fluorescence in arbitrary units (a.u.)×10⁴.

FIG. 9 shows results from assays using a tandem sfCherry11 tag in the reverse primer to generate a red fluorescence output signal for detection of mutations in the BRCA1 and BRCA2 genes: 3029_3030del, 68_69del and 2679 2682del of BRCA1 and 4936_4939del of BRCA2. Synthetic wild-type and homozygous mutant DNA samples were used as mock templates. On the x-axis is time in minutes and on the y-axis is fluorescence in arbitrary units (a.u.).

FIG. 10 shows the fluorescence output from an assay detecting the CFTR W1282X mutation that is associated with cystic fibrosis. Synthetic wild-type and homozygous mutant DNA samples were used as mock templates. On the x-axis is time in minutes and on the y-axis is fluorescence in arbitrary units (a.u.).

FIG. 11 shows the green fluorescence output from an assay detecting a mutation that causes Thiamine Metabolism Dysfunction syndrome 2 (THMD2). Synthetic wild-type and homozygous mutant DNA samples were used as mock templates. On the x-axis is time in minutes and on the y-axis is fluorescence in arbitrary units (a.u.).

FIG. 12 shows the green fluorescence output from assays designed to generate fluorescence when detecting the mutations 1086_1141del, 68_69del in BRCA1 genomic DNA (gDNA) and 658_659del in BRCA2 gDNA. Synthetic wild-type and homozygous mutant DNA samples were used as mock templates. On the x-axis is time in minutes and on the y-axis is fluorescence in arbitrary units (a.u.).

FIG. 13 shows the dual green fluorescence (WT sequence present) (top) and red fluorescence (assay control) signals (bottom) from assays designed to detect the mutations 5266dup, 68_69del in the cDNA of the BRCA1 gene and 658_659del in the cDNA of the BRCA2 gene. Synthetic wild-type and homozygous mutant DNA samples were used as mock templates. On the x-axis is time in minutes and on the y-axis is fluorescence in arbitrary units (a.u.).

FIG. 14 shows the dual green fluorescence (WT sequence present) (top) and red fluorescence (assay control) signals (bottom) from assays designed to detect the mutations 2475del, 1086_1141del in the cDNA of the BRCA1 gene and 4936_4939del in the cDNA of the BRCA2 gene. Synthetic wild-type and homozygous mutant DNA samples were used as mock templates. On the x-axis is time in minutes and on the y-axis is fluorescence in arbitrary units (a.u.).

FIG. 15 shows the dual green fluorescence (WT sequence present) (top) and red fluorescence (assay control) signals (bottom) from assays designed to detect the mutations 2475del, 3029_3030del and 2679_2682del in cDNA of the BRCA1 gene using optimized amplification primers. Synthetic wild-type and homozygous mutant DNA samples were used as mock templates. On the x-axis is time in minutes and on the y-axis is fluorescence in arbitrary units (a.u.)×10³.

### DETAILED DESCRIPTION

All publications, including but not limited to patents and patent applications, cited in this specification are herein incorporated by reference as though set forth in their entirety in the present application.

Provided herein are methods of detecting target nucleic acids in a sample. The methods are rapid, reliable, low cost, and sensitive enough to detect low concentrations of target nucleic acids. The detection of a target nucleic acid may indicate the presence of a pathogen, genetic marker, disease condition, genetic predisposition, or other information. Some of these methods can be performed in the absence of complex equipment and biosafety precautions, such as, using a single portable assay device.

Provided herein is a method of detecting a target nucleic acid in a sample, the method comprising an amplifying step (a) using an amplification primer to append translation elements and a peptide reporter encoding sequence to a nucleic acid amplification product comprising a target nucleic acid, a translating step (b) to incorporate a peptide reporter into a translation product of the target nucleic acid, and a detecting step (c) to detect the presence of the translated target nucleic acid by detecting the peptide reporter. In some embodiments, the method comprises or consists essentially of the steps of: (a) amplifying a nucleic acid obtained from a sample using a first peptide primer and a second peptide primer, wherein the first peptide primer is a forward primer comprising, from 5′ to 3′, (1) a nucleic acid sequence encoding a ribosome binding site (RBS), (2) a start codon, and (3) a nucleic acid sequence complementary to a first portion of a target nucleic acid sequence, and wherein the second peptide primer is a reverse primer comprising, from 3′ to 5′, (1) a nucleic acid sequence complementary to a second portion of the target nucleic acid sequence, (2) a reverse complement of a sequence encoding a peptide reporter, and (3) a reverse complement of a stop codon in-frame with the reverse complement of the sequence encoding the peptide reporter; (b) translating the amplified nucleic acid into translation products using a cell-free translation reaction; and (c) detecting the translated peptide reporter among the translation products.

In some embodiments, the target nucleic acid sequence comprises a protein coding sequence spanning the first and second portions and in-frame with the start codon of the first peptide primer and in-frame with the complement of the reverse complement sequence encoding the peptide reporter. In other embodiments, the target nucleic acid sequence comprises at least one open reading frame having a start codon in-frame with the sequence encoding the peptide reporter. In some embodiments, the target nucleic acid sequence comprises an open reading frame which begins 3′ of the first portion but 5′ of the second portion and in-frame with the sequence encoding the peptide reporter.

The first portion and the second portion of the target nucleic acid sequence of the primers allow for amplification of a target sequence that comprises a coding sequence positioned in-frame with the start codon of the first peptide primer and/or the sequence (i.e. the complement of the reverse complement of the peptide reporter of the second primer) encoding the peptide reporter of the second peptide primer. In some embodiments, the first portion of the target nucleic acid sequence comprises a protein coding sequence positioned in-frame with the start codon of the first peptide primer and the sequence encoding the peptide reporter. In some embodiments, the second portion of the target nucleic acid sequence comprises a protein coding sequence positioned in-frame with the sequence encoding the peptide reporter and in-frame with the start codon of the first peptide primer after amplification.

The term “detecting,” “detect” or “detection” as used herein indicates the determination of the existence or presence of a target molecule or signal in a limited portion of space, including but not limited to a sample, a reaction mixture, a molecular complex and a substrate including a platform and an array. Detection is “quantitative” when it refers, relates to, or involves the measurement of quantity or amount of the target molecule or signal (also referred to as quantitation), which includes but is not limited to any analysis designed to determine the amounts or proportions of the target or signal. Detection is “qualitative” when it refers, relates to, or involves identification of a quality or kind of the target or signal in terms of relative abundance to another target or signal, which is not quantified. An “optical detection” indicates detection performed through a detectable light signal(s): fluorescence, spectra, or images from a peptide reporter of interest or a probe bound to the peptide reporter during the detecting.

In some embodiments, the method comprises contacting the translation products with an agent (e.g., a reagent or detectable agent) which binds the peptide reporter and allows for detection, either directly or indirectly, of the peptide reporter via a detectable agent. In some embodiments, the contacting occurs during the translating step or detecting step. In some embodiments, the contacting occurs after the translating step and before the detecting step.

As used herein, the term “detectable” means the molecule or atom can be detected by a known process, whether directly or indirectly, such as, after a processing step, chemical reaction, enzymatic reaction, and/or the addition of an additional reagent(s). For example, a “detectable peptide reporter,” is a peptide which can be detected by a known process, whether directly or indirectly, such as via an agent that specifically binds the peptide. In some embodiments, the agent is a detectable agent, whereas in other embodiments the agent need not be detectable in isolation. Instead, the agent need only be detectable in the presence of a peptide reporter, whereby a complex between the agent and peptide reporter can form, together representing a detectable agent. In some embodiments, the agent is differentially detectable after binding to the peptide reporter to form a detectable agent that differs in detection characteristics of either the agent alone or the peptide reporter alone.

In some embodiments, the method comprises contacting the translation products with an agent which binds the peptide reporter and allows for detection of the peptide reporter. In some embodiments, the agent binds to the peptide reporter to form a detectable agent comprising the peptide reporter (e.g. an assembled split reporter). The agent may be, e.g., a component of a cell-free translation reaction or brought into contact with the peptide reporter after a translating step.

As used herein, a “detectable agent” is an agent other than the peptide reporter (but optionally comprising all or a portion of the peptide reporter) that can be detected by a known process from a composition of translation products. In some embodiments, the detectable agent which binds the peptide reporter is an antibody or antibody fragment. In some embodiments, the agent which binds the peptide reporter can form a complex of the peptide reporter bound by the agent wherein the complex is fluorescent, luminescent, and/or capable of catalyzing a colorimetric reaction. In some embodiments, the agent which binds the peptide reporter is a protein, such as, e.g., a GFP1-10, sfCherry1-10, or lacZ-ω protein. A detectable agent may be detected by a method known to the skilled worker and/or described herein. For example, detecting a detectable agent or detectable peptide reporter may be accomplished by spectroscopic, photochemical, biochemical, immunochemical, electrical, optical, or chemical means. Detectable agents include, for example, fluorescent dyes, radioisotopes, enzymes, and colorimetric labels such as colloidal gold, silver, selenium, or other metals, and antibodies and antibody fragments comprising or conjugated to any of the aforementioned. Antibodies and antibody fragments which do not comprise or are not conjugated to a label are detectable agents in the sense that they can readily be detected with a secondary agents, such as, e.g. a secondary antibody or protein A, protein G, or protein L ligand-probe.

In some embodiments, the agent is a detectable agent which binds to the peptide reporter thereby allowing for detection of the peptide reporter via the complex formed by the detectable agent bound to the peptide reporter. In some embodiments, the detectable agent which binds the peptide reporter comprises an antibody or antibody fragment which binds the peptide reporter.

In some embodiments, the translating step (b) comprises a cell-free translation reaction comprising one or more reagents that bind to the peptide reporter as it is translated or after it is translated to form at least one detectable agent that allows detection of the peptide reporter, and the detecting step (c) comprises detecting the peptide reporter by detecting the detectable agent. In some embodiments, the agent is a detectable agent comprising an antibody or antibody fragment which binds the peptide reporter and the detecting step (c) comprises detecting the peptide reporter by detecting the detectable agent.

In some embodiments, the detecting step (c) comprises detecting a detectable agent, wherein the detectable agent is bound to peptide reporter or wherein the detectable agent is formed by the combination or complex of the peptide reporter bound by another agent. In some embodiments, the detectable agent is fluorescent, luminescent, or capable of catalyzing a colorimetric reaction. In some embodiments, the detectable agent comprises a radioisotope.

The translating step can comprise using a cell-free translation reaction or a transcription-translation reaction. If a cell-free transcription-translation reaction will be used then the forward peptide primer further comprises 5′ to the RBS a transcriptional promoter, which drives the transcription of the amplified nucleic acid to produce a target sequence-peptide reporter mRNA and translation of the target sequence-peptide reporter mRNA into a translated target sequence peptide reporter. The peptide reporter is detected in the detecting step (c) indicating the presence of the target nucleic acid in the sample. In some embodiments, the transcriptional promoter comprises a T7 RNA polymerase promoter. In some embodiments, the RBS is prokaryotic, the start codon is prokaryotic, and/or the stop codon is prokaryotic.

In some embodiments, the peptide reporter comprises, or can be made to comprise with the addition of an agent, a fluorescent reporter or colorimetric reporter. The peptide reporter can be selected from a fluorescent reporter and colorimetric reporter. The peptide reporter can comprise a GFP11 peptide, lacZ-α peptide, sfCherry11 peptide, or sfCherry₂11 peptide. The fluorescent reporter can comprise a GFP11 peptide. When the peptide reporter is a GFP11 peptide, then step (c) comprises contacting the translation products with an agent comprising a GFP1-10 protein which binds to the peptide reporter to form a fluorescent detectable agent. The colorimetric reporter can comprise a lacZ-α peptide. When the peptide reporter comprises a lacZ-α peptide, then step (c) of the method comprises contacting the translation products with a detectable agent comprising LacZ-ω protein which binds to the peptide reporter, and the detectable agent is detected by a colorimetric reaction comprising chlorophenol red-β-D-galactopyranoside. The colorimetric reporter can comprise a sfCherry11 peptide or a sfCherry₂11 peptide. When the peptide reporter comprises a sfCherry11 peptide, then step (c) comprises contacting the translation products with an agent comprising sfCherry1-10 protein which binds to the peptide reporter to form a fluorescent detectable agent. When the peptide reporter comprises a sfCherry₂11 peptide, then step (c) comprises contacting the translation products with an agent comprising sfCherry₂1-10 protein which binds to the peptide reporter to form a fluorescent detectable agent.

For some embodiments, the method can indicate the presence of the target nucleic acid in a sample at a concentration as low as 90 attomolar (aM), 80 aM, 70 aM, 60 aM, 50 aM, 40 aM, or 30 aM.

The amplifying step can comprise a polymerase chain reaction (PCR). In some embodiments, the amplifying is isothermal. The amplifying step can comprise a recombinase polymerase amplification (RPA) or reverse-transcription recombinase polymerase amplification assay (RT-RPA).

In some embodiments, the method is performed in less than 6 hours. In some embodiments, the method is performed in less than 6 hours, in less than 3 hours, in less than 2 hours, in less than 1 hour, or in less 30 minutes. The sample can be a biological sample obtained from a subject. The subject can be a human subject.

In some embodiments, the method comprises or consists essentially of the steps of: (a) amplifying a nucleic acid obtained from a sample using a first peptide primer and a second peptide primer, wherein the first peptide primer is a forward primer comprising, from 5′ to 3′, (1) a nucleic acid sequence encoding a ribosome binding site (RBS), (2) a start codon, (3) a nucleic acid sequence encoding a second peptide reporter in-frame with the start codon, and (4) a nucleic acid sequence of a first portion of a target nucleic acid sequence, and wherein the second peptide primer is a reverse primer comprising, from 3′ to 5′, (1) a nucleic acid sequence complementary to a second portion of the target nucleic acid sequence, (2) a reverse complement of a sequence encoding a first peptide reporter, and (3) a reverse complement of a stop codon in-frame with the reverse complement of the sequence encoding the first peptide reporter; wherein the target nucleic acid sequence amplified in step (a) comprises a protein coding sequence spanning the first and second portion of the target nucleic acid sequences in frame with the start codon and the amplified product also comprises the sequence encoding the second peptide reporter in frame with the start codon and the sequence encoding the second peptide reporter; (b) translating the amplified nucleic acid into translation products using a cell-free translation reaction; and (c) detecting the translated second peptide reporter among the translation products. In some embodiments, the amplified product may also comprise the first peptide reporter and if the first peptide reporter is in frame with the target nucleic acid sequence after the translation step, then the detecting step (c) further comprises detecting the translated first peptide reporter. If the first peptide reporter is not in frame with the target sequence, then after the translating step the first peptide reporter will not be detected. Thus one of skill in the art can determine if there is a mutation affecting the reading frame or translation of the target sequence. If the second peptide reporter is detected, but the first peptide reporter is not, then a mutation must be present in the target nucleic acid in the sample that affects the reading frame of the target sequence, such as a frame shift or nonsense mutation.

The target nucleic acid can comprise a single nucleotide polymorphism (SNP) or single nucleotide variant (SNV), and the forward primer can comprise one or more nucleotides corresponding to a SNP or SNV of interest within the nucleic acid sequence of the first portion of the target nucleic acid sequence. The detecting step (c) can indicate the presence in the sample of a target nucleic acid comprising a SNP or SNV of interest. In some embodiments, the nucleotide corresponding to the SNP or SNV is positioned within 10 nucleotides of the 3′ end of the first peptide primer. In some embodiments, wherein the nucleotide corresponding to the SNP or SNV is positioned within the first ten, seven, six, five, four, three, two or one nucleotides of the most 3′ nucleotides of (a) the nucleic acid sequence of a first portion of the target nucleic acid or (b) the nucleic acid sequence complementary to the second portion of the target nucleic acid sequence. In one embodiment, the nucleotide corresponding to the SNP or SNV is at the 3′ end or within one nucleotide of the 3′ end of one of the two primers.

The methods and compositions provided herein are based at least in part on the inventors' development of a platform for nucleic acid detection that uses peptide reporter primers based on the target nucleic acid of interest. The primers append translation signal elements and a peptide reporter to the target sequence following amplification. Amplified product can then be added to a cell-free translation reaction in a test tube or on a paper substrate for detection via fluorescence or color output. In some embodiments, the primers append transcription-translation signal elements and a peptide reporter to the target sequence following amplification. Amplified products can then be added to a cell-free transcription-translation reaction in a test tube or on a paper substrate for detection via fluorescence or color output. Another embodiment comprises a dual reporter output system for viral strain identification and/or to detect mutations such as single-nucleotide variants (SNVs) that produce stop codons or frame-altering (e.g., nonsense or frameshift) mutations.

By way of example, it is demonstrated herein that the methods and compositions are useful to detect SARS-CoV-2 and to distinguish SARS-CoV2 from other closely related SARS-CoV viruses, to detect mutations in cancer-causing genes (e.g., breast cancer gene 1 (BRCA1) and breast cancer gene 2 (BRCA2)), to detect drug resistance-inducing mutations (e.g., single nucleotide mutations related to a HIV drug resistance gene), and to differentiate homozygous and heterozygous mutations. An advantage of the methods described herein is that they can be applied for the detection and identification of essentially any nucleic acid-containing organism. Accordingly, the pathogen can be virtually any pathogen or infectious agent (e.g., viruses, parasites, bacteria, fungi, and prions) for which genetic information is available. Further advantages of the methods and compositions of this disclosure include, without limitation, low cost tests for which the results are obtained quickly and easy to interpret. Depending on the sample concentration and cell-free transcription-translation reaction speed, results can be obtained in about 1 hour. Thermocycling equipment is not required because the methods may employ isothermal amplification methods, such as recombinase polymerase amplification (RPA) and reverse-transcription recombinase polymerase amplification (RT-RPA).

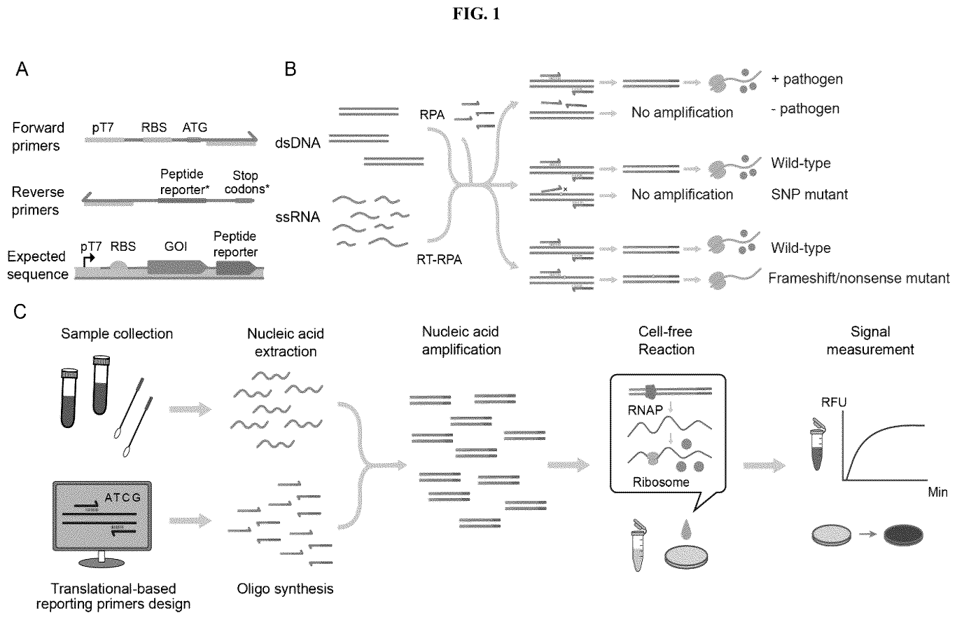

Accordingly, in a first aspect, provided herein are methods and compositions to detect a target nucleic acid. As illustrated in FIG. 1A, the platforms use peptide reporter primers. In some embodiments, the primers append transcription-translation signal elements and a peptide reporter to the target sequence following amplification. Amplified product can then be added to a cell-free transcription-translation reaction in a test tube or on a paper substrate for detection via fluorescence or color output.

In some embodiments, a method for detecting a target nucleic acid in a sample comprises or consists essentially of: (a) amplifying nucleic acids obtained from a biological sample of a subject using a first peptide primer and a second peptide primer. In some embodiments, the first peptide primer is a forward primer comprising, from 5′ to 3′, an optional nucleic acid sequence encoding a promoter sequence (e.g. T7 RNA polymerase promoter), a ribosome binding site (RBS), a start codon, and a nucleic acid sequence of a first portion of the target nucleic acid sequence. In some embodiments, the second peptide primer is a reverse primer comprising, from 5′ to 3′, a reverse complement of a stop codon, a reverse complement of a sequence encoding a peptide reporter and a nucleic acid sequence complementary to a second portion of the target nucleic acid sequence, in which the target sequence, the peptide reporter sequence and the stop codon are in frame. The amplified product will optionally comprise a promoter. The amplified product will comprise a RBS, a start codon in frame with an amplified target sequence in frame with a peptide reporter and followed by an in frame stop codon. The method may further comprise (b) adding the amplified nucleic acids to a cell-free translation reaction or where the promoter was included in the amplified product the amplified nucleic acids can be added to a cell-free transcription-translation reaction. Finally after cell-free translation, the method may further comprise step (c) detecting the peptide reporter among products of the cell-free translation reaction, where the presence of the peptide reporter indicates the presence of the target nucleic acid in the sample. In this manner, samples with the target nucleic acid can have their target nucleic acid amplified by the peptide primers to produce peptide reporter signal output while samples without target nucleic acid cannot. The methods may be useful to detect the presence of a pathogen, SNP, SNV, and/or frame altering mutation in the sample.

In some embodiments, confirmation of the presence or sequence of an amplification product can be achieved by altering primer specificity to only allow binding and amplification of a target sequence in a sample, which produces signal output. In general, the sequences within the primers that bind to the target nucleic acid sequence follow the same strategies used for designing primers for the particular amplification method to be used in the assay (e.g., PCR, RPA, or NASBA). For instance, PCR primer binding sites are designed to have the correct melting temperature for the chosen thermal cycling profile, to have minimal secondary structure and to have low homology with other known sequences (e.g., sequencing screening using BLASTn). RPA primers are designed to bind to regions 30 to 36 nucleotides (nts) in length to ensure that recombinase-primer complexes form and invasion into the double-stranded DNA template occurs efficiently. Because peptide primers may contain many sequences that are not conserved, it can be advantageous to implement custom primer design algorithms to optimize the secondary structure of the primers, the choice of codons used in encoding the reporter peptide, and the probability of primer dimer formation. In the algorithm, non-conserved, untranslated bases in the primers are refined using the NUPACK software package to minimize the overall secondary structure of the peptide primer. Coding sequences are subjected to a Monte-Carlo method that uses synonymous codons to iteratively improve the secondary structure of the peptide primer while selecting codons with the highest usage frequency in the E. coli genome, because the cell-free reactions employ E. coli components. Following repeated cycles of optimization of the sequences of the untranslated regions and the translated regions, the primers are also screened for primer dimer formation probability. The algorithm can yield peptide primers with excellent performance; however, functional primers can be obtained without using any custom software tools.

The size of the reporter peptides incorporated into the primers is limited by two factors.

First, the size of oligonucleotides available at reasonable costs and synthesis fidelity is currently limited to ˜200 nts, which means that a peptide of ˜60 residues is the longest that can be conveniently added through amplification. Secondly, longer primers can reduce the sensitivity of the amplification process and bind to off-target sites and other primers in the reaction. Some of these effects can likely be reduced using peptide primer design algorithms, but this phenomenon does make the use of longer primers more challenging.

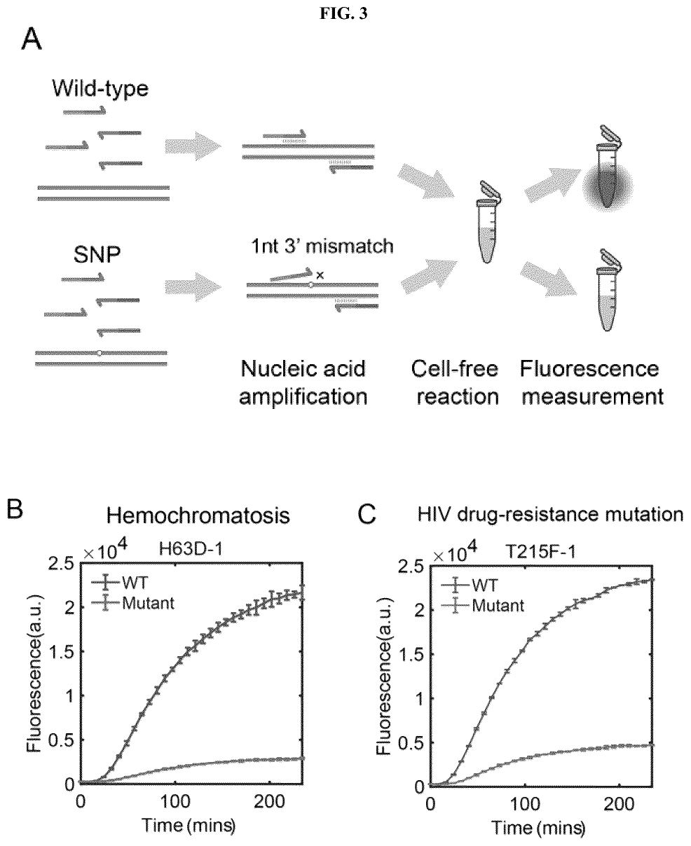

In some embodiments, the peptide primers are designed to detect one or more single nucleotide polymorphisms (SNP) in a sample. For instance, at least one of the peptide primers can comprise one or more 3′ single nucleotide mismatches relative to the wild-type nucleic acid sequence such that the detection methods detect single nucleotide variants as illustrated in FIG. 3A-C. In such cases, production of the detectable reporter peptide only occurs when the sample contains the SNP of interest. The reporter peptide is not expressed when the sample does not contain the SNP of interest. In this manner, the methods of this disclosure are useful to detect many genetic markers or genetic predispositions to disease that are due to one or more single nucleotide polymorphisms.

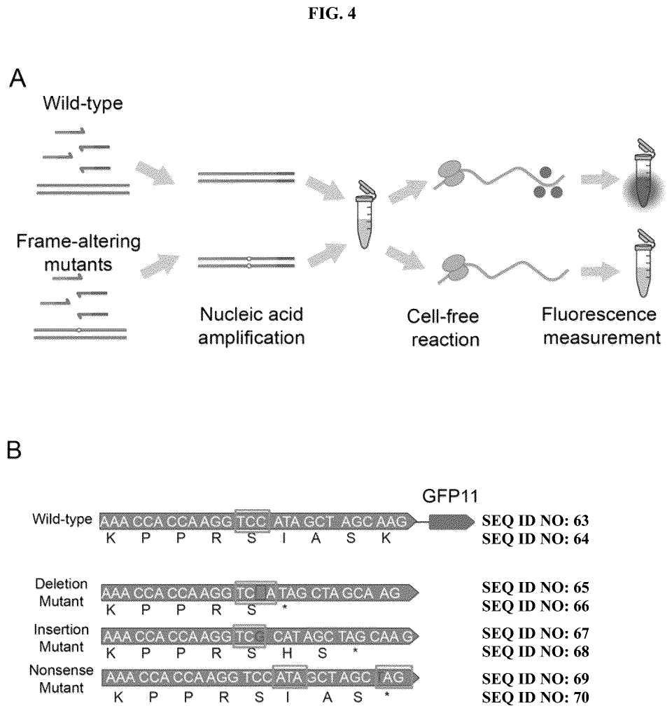

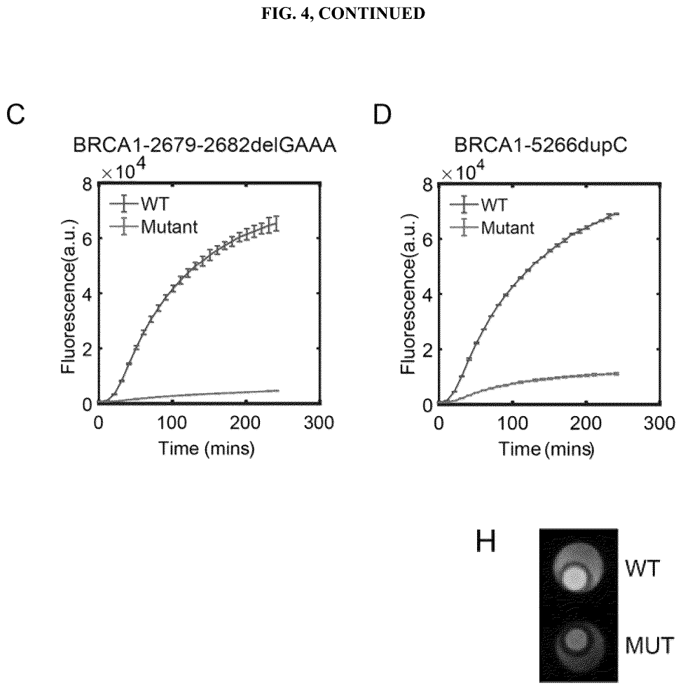

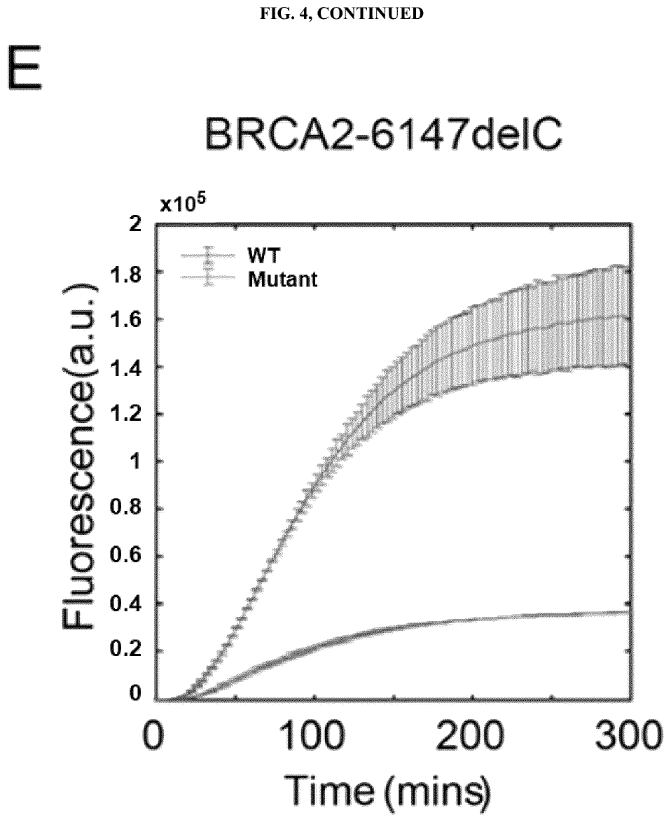

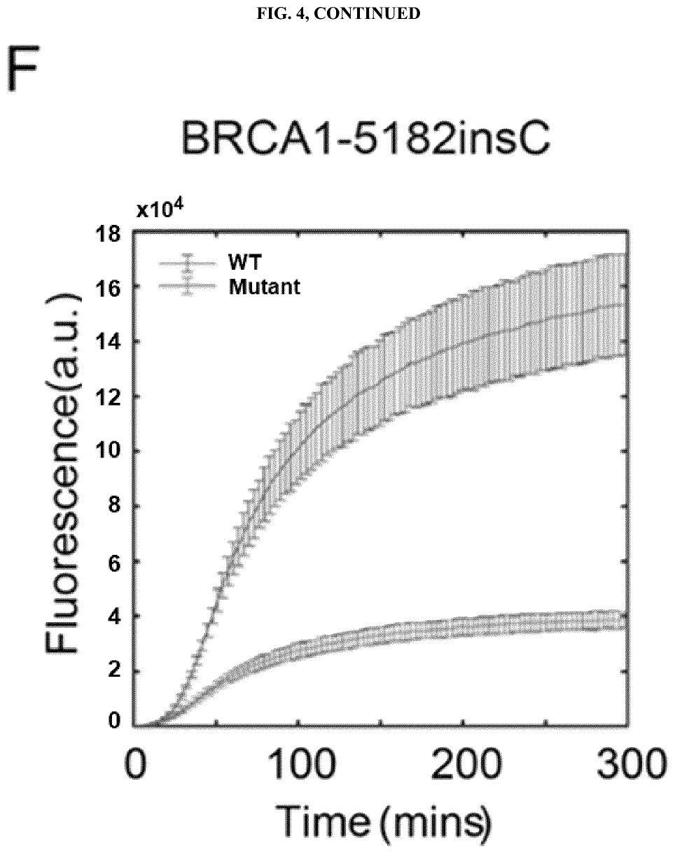

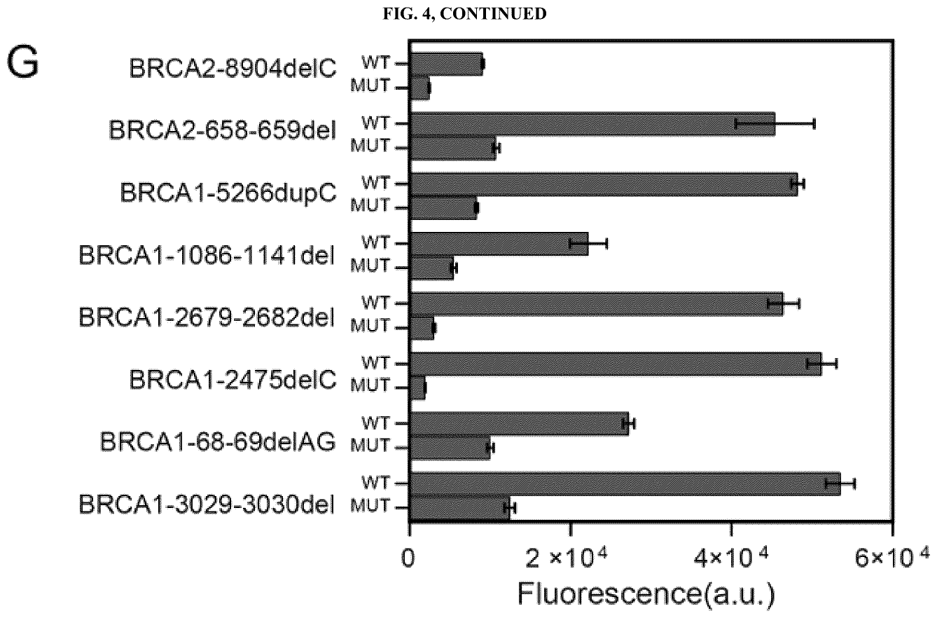

In some embodiments, the peptide primers are designed to detect one or more frame-shift or nonsense mutations. As used herein, the term “frame-shift mutation” (also known as a “frame-alternating mutation”) refers to genetic mutation caused by indels, i.e., an insertion or deletion of a number of nucleotides that is not evenly divisible by three from a DNA coding sequence, leading to an alteration in the codons of the following sequence and, hence, the final gene product. As used herein, the term “nonsense mutation” refers to a nucleotide change which changes a codon that specified an amino acid to one of the stop codons (TAA, TAG, or TGA) and, hence, leads to a truncated final gene product. As illustrated in FIG. 4A-B, both wild-type and mutant samples can be amplified by primers, but only samples having a valid open reading frame (an open reading frame extending through the reverse primer's target nucleic acid homology sequence) will express reporter peptides. By way of example, peptide reporter primers can be designed to have a downstream reporter peptide sequence in-frame with the wild-type sequence, thus allowing subsequent expression of the reporter peptide. In this manner, the methods of this disclosure are useful to detect many genetic markers or genetic predispositions to disease that are due to a nonsense mutation(s), a frame shift mutation(s) or to an out of frame deletion(s).

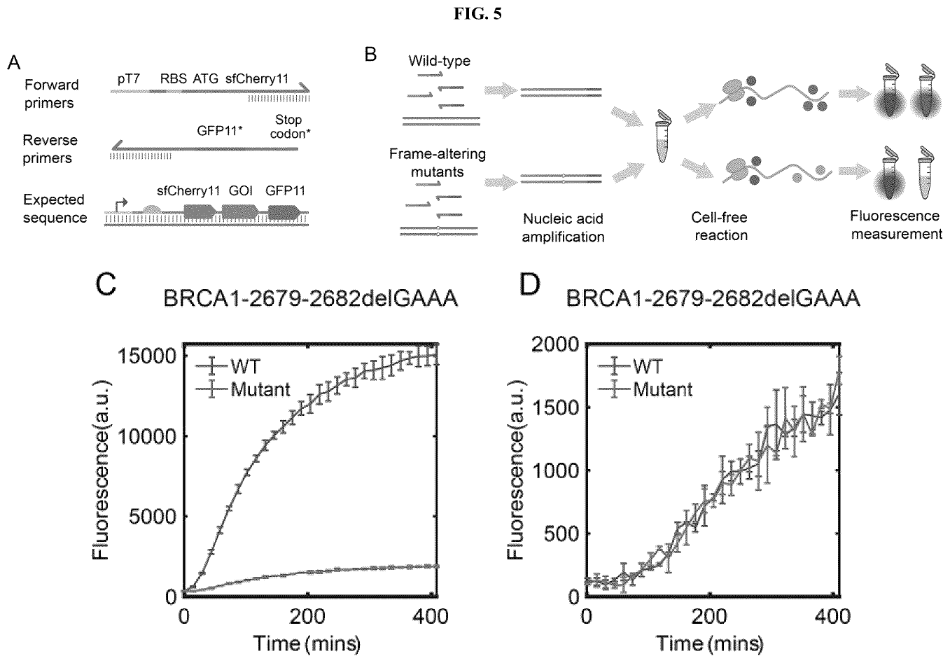

Referring to FIG. 5A-B, peptide reporter primers are designed in some cases to detect frame-alternating mutations. By way of example, the peptide reporter can be a fluorescent peptide such as sfCherry11. A sequence encoding the peptide reporter sequence can be inserted into the forward primers permitting translation of the peptide reporter even if the downstream reading frame contains a stop codon. When two different peptide reporters are used as illustrated in FIG. 5B, the platform contains a self-calibrating feature (i.e., its own internal positive control). For example, both wild-type and mutant samples can be amplified by primers and will allow expression of a peptide reporter included upstream of the target sequence in the amplified product to produce one type of fluorescence (red fluorescence in this example), but only samples having wild-type open reading frames for the target sequence will express a peptide reporter included downstream of the target sequence for production of a different type of fluorescence (green fluorescence in this example).

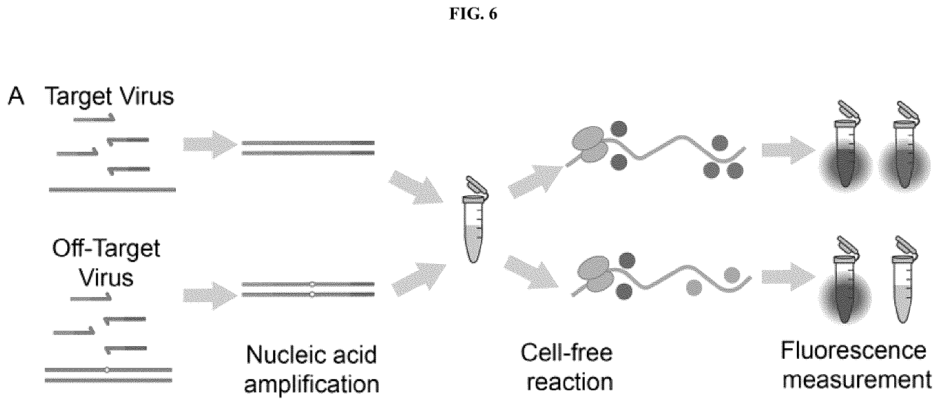

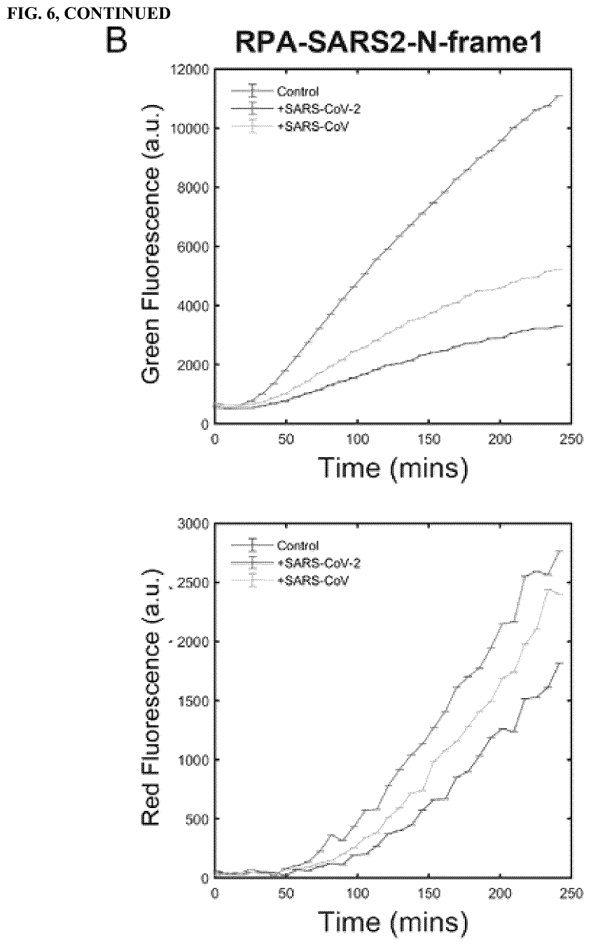

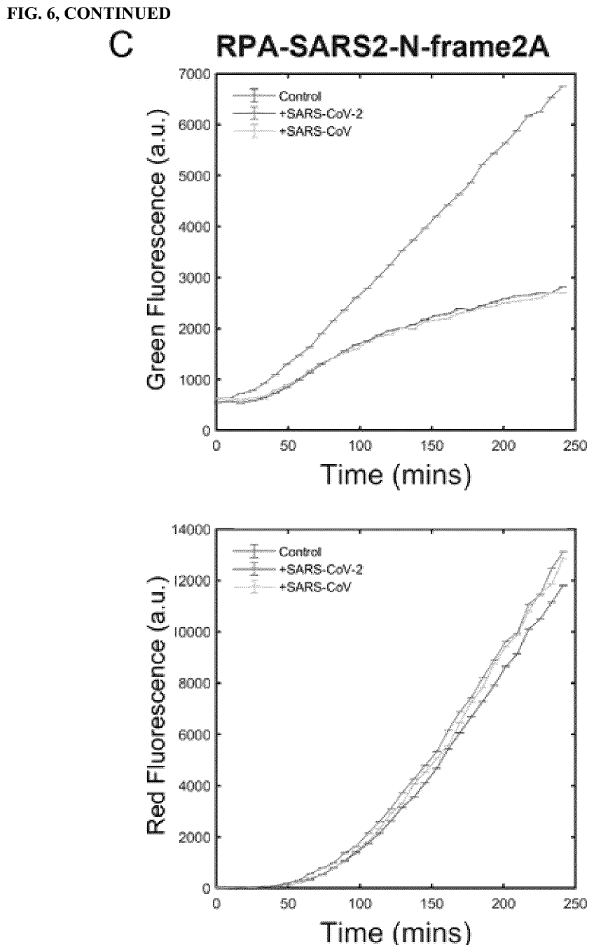

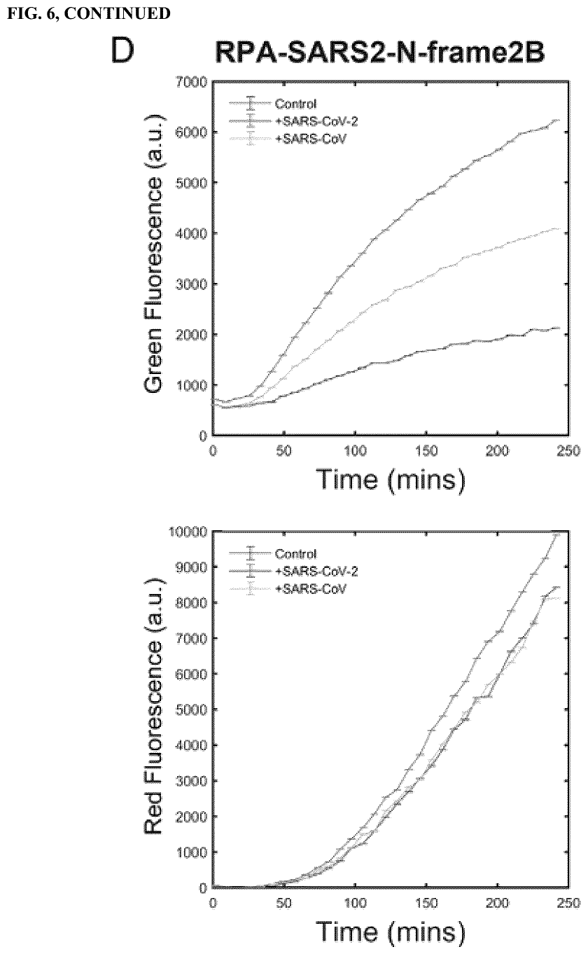

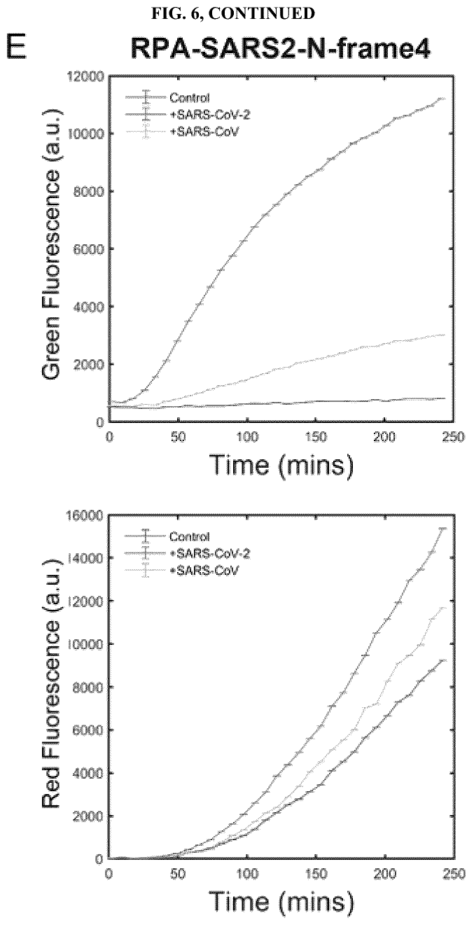

In some cases, peptide reporter primers are designed to provide a reporter output system for identification of a pathogen or pathogen strain, subtype, or variant (e.g. a pathogenic microbe). As illustrated in FIG. 6A, peptide reporter primers can be designed for dual fluorescence output. In some embodiments, for example, two different fluorescence proteins are used to distinguish between two closely related pathogens (e.g., closely related virus strains, bacterial strains, fungal strains, parasites, pathogen subtypes, pathogen variants, etc.). Both the target pathogen and a closely related pathogen can be amplified by primers and will allow the production of a first type of fluorescence, but only samples having open reading frame from the correct virus will express the second reporter peptide for a second type of fluorescence. In this manner, the methods of this disclosure are useful to detect and distinguish pathogen nucleic acids, such as distinguishing between related bacterial, fungal, parasite, or viral strains (e.g., SARS-CoV and SARS-CoV-2).

Any appropriate peptide reporter can be used for the methods described herein. For instance, the peptide reporter can be a fluorescent reporter or a colorimetric reporter. Illustrative but non-limiting reporter proteins include lacZ, catalase, xylE, GFP, RFP, YFP, CFP, neomycin phosphotransferase, luciferase, mCherry, and derivatives or variants thereof.

As used herein, the term “fluorescent” refers to protein or protein complex that has the ability to emit light of a particular wavelength (emission wavelength) when exposed to light of another wavelength (excitation wavelength). Non-limiting examples of fluorescent proteins include the green fluorescent protein (GFP; see, for instance, GenBank Accession Number M62654) and natural and engineered variants thereof. Fluorescent proteins with distinct excitation and emission properties are familiar to the skilled artisan; for example, functional GFPs, RFPs, CFPs and YFPs comprise distinct excitation and emission properties.

In some embodiments, the peptide reporter is part of a split reporter system, such as a split fluorescent or split colorimetric reporter system. In some embodiments, the peptide reporter represents a split reporter system. A split reporter system divides a detectable reporter into two or more separate structural components. On their own, the split report components are not detectable by the method used to detect the assembled combination of the components. When the split components are combined (e.g. brought in contact with each other in solution), the components spontaneously self-assemble to produce a detectable agent. Often in paired components, one component of the split pair is often relatively small (e.g. about 8 to 30 amino acid residues) whereas the other component can be, e.g., as large as 200 to 1000 amino acid residues.

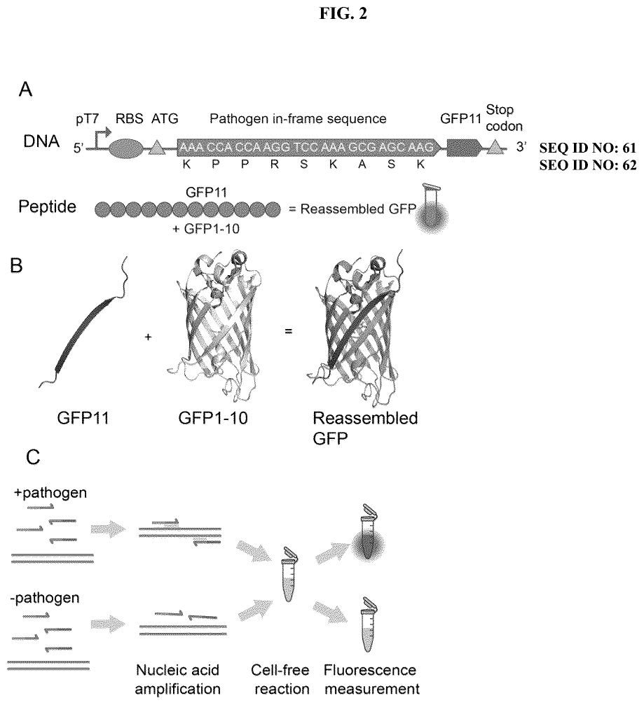

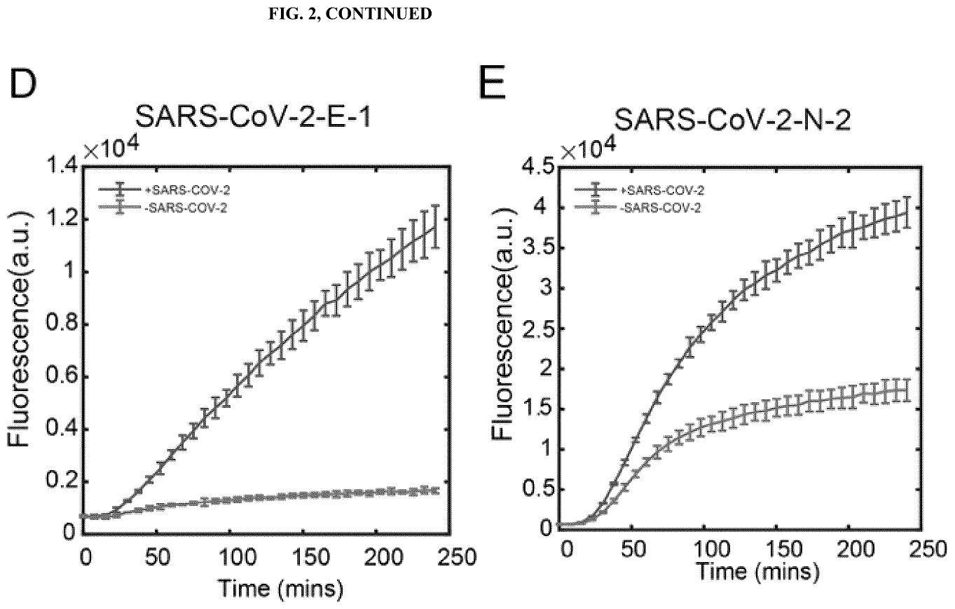

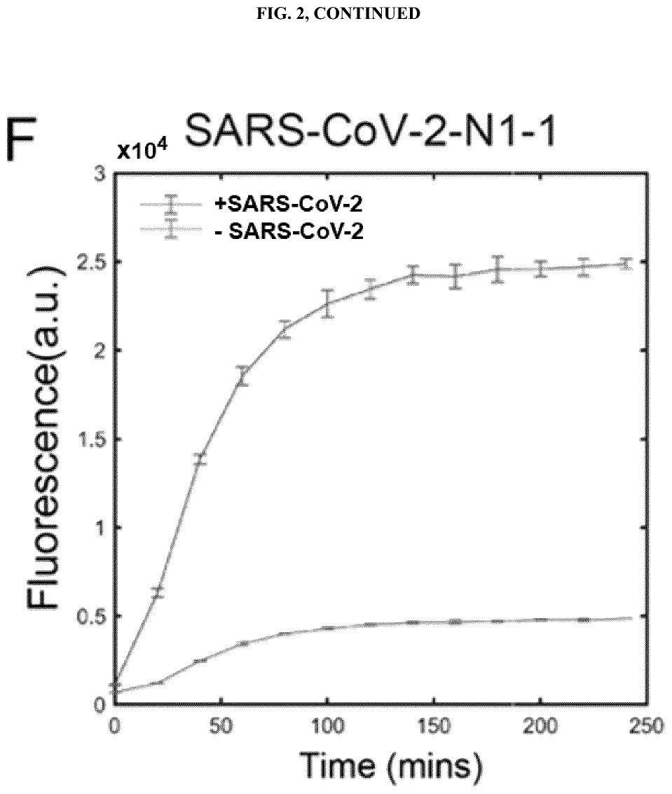

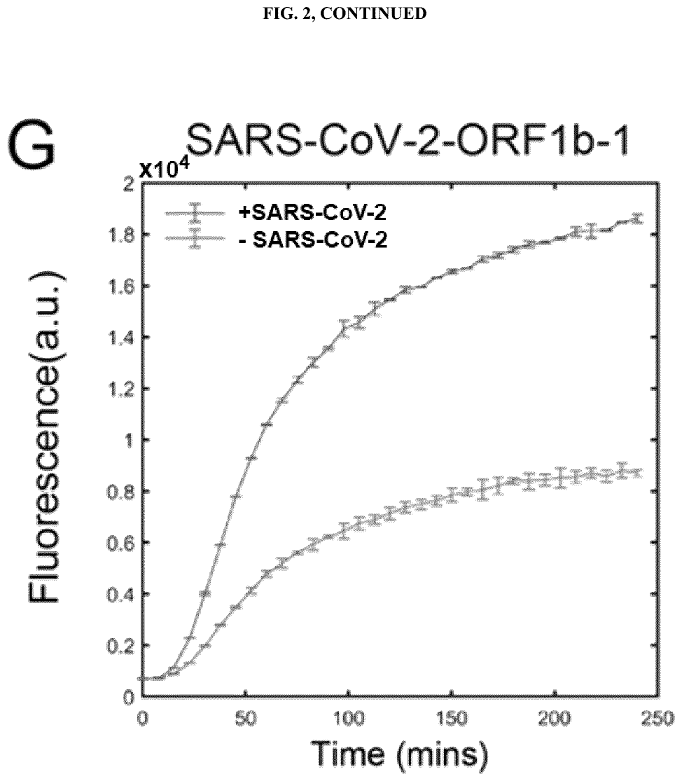

In some embodiments, the peptide reporter is a fluorescent peptide as part of a split reporter system, such as the GFP1-10/GFP11 split reporter system as illustrated in FIG. 2A-B. GFP11, which is a short 16-amino-acid peptide, is non-fluorescent on its own but will self-assemble with the GFP1-10 protein to produce green fluorescence. Referring to FIG. 2C-D, GFP11 is used as the reporter protein. In some cases, expression of GFP11 only occurs in the presence of the target nucleic acid in the sample. When the cell-free transcription-translation reaction can comprise GFP1-10 protein, GFP11 expressed in the presence of the target nucleic acid will self-assemble with GFP1-10 to produce green fluorescence. While the GFP1-10/GFP11 split reporter system is exemplified in this disclosure, other split reporter systems can be used, including split non-fluorescent proteins and split non-colored chromoproteins.

In some embodiments, the peptide reporter is a colorimetric reporter such as a chromoprotein or enzyme catalyzing a colorimetric reaction. The term “colorimetric” is defined as an analysis where the reagent or reagents constituting the peptide reporter system produce a color change in the presence or absence of an analyte. The degree the color changes in response to the analyte (e.g., target nucleic acid) may be quantified by colorimetric quantification methods known to those of ordinary skill in the art. In some embodiments, standards containing known amounts of the selected analyte may be analyzed in addition to the sample to increase the accuracy of the comparison.

Chromoproteins, proteins which exhibit a visible color, can also be used as colorimetric reporters. Illustrative but not-limiting chromoprotein reporters include meffRed, fwYellow, amilGFP, cjBlue, meffBlue, aeBlue, and tsPurple.

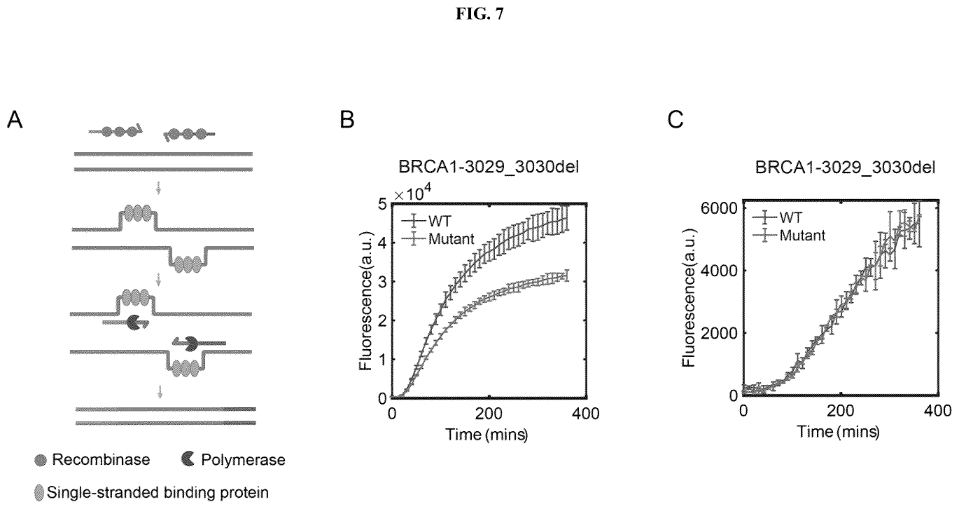

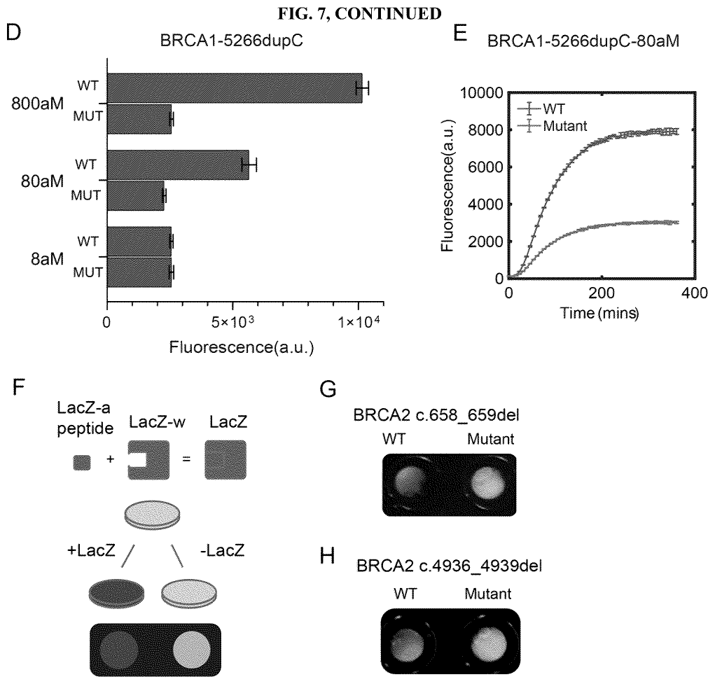

In some embodiments, the peptide reporter is a colorimetric reporter which is an enzyme catalyzing a colorimetric reaction, such as a galactosidase. The lacZ gene which encodes β-galactosidase has been widely used as colorimetric reporters and has been shown to work in paper-based platforms. As demonstrated herein, a 39-residue lacZ-α peptide can be used as the peptide reporter to provide a colorimetric readout. Referring to FIG. 7F, lacZ-α peptides will reassemble with lacZ-ω protein (e.g., supplied in the cell-free system) to form functional β-galactosidase, which catalyzes a colorimetric reaction, cleavage of the initially yellow substrate chlorophenol red-β-D-galactopyranoside to purple. By using lacZ-α peptide reporter, the readout can be colorimetric, and the detection can be paper-based. This will further facilitate easy reading of the results, and the detection assay can be deployable and suitable for point-of-care, at-home, or in the field use.

In some embodiments, the cell-free transcription-translation reaction comprises one or more reagents that binds to the peptide reporter to form a fluorescent or colored product or to catalyze a colorimetric reaction. In some embodiments, the reagent is a component of a split reporter, such as a complementary component to the peptide reporter which is capable of binding to the peptide reporter to form an assembled detectable agent.

The methods of this disclosure, which combine peptide reporter primers, isothermal amplification, and cell-free transcription-translation reactions, are highly sensitive and can reach attomolar (aM) detection limits and lower. For instance, the methods are useful to detect a target nucleic acid present in a sample at a concentration as low as 50 aM, 25 aM, 10 aM, or 5 aM. To further lower the detection limit of these methods, it can be advantageous to use multiple peptide reporter primers that bind along different portions of a target nucleic acid. In addition, an initial amplification reaction with conventional primers lacking transcription-translation or peptide signals can be used to lower the detection limit.

In some embodiments, the method is performed in less than about 6 hours, 5 hours, 4 hours, 3 hours, 2 hours, or 1 hour. In some embodiments, the method is performed in less than about 60 minutes, 50 minutes, 40 minutes, 30 minutes, 20 minutes, or 15 minutes. Expression of the peptide reporter, if present, can be detectable in less than 6 hours. Expression of the peptide reporter, if present, can be detectable in less than 30 minutes. In some embodiments, the peptide reporter, if present, is detectable in less than 6 hours (e.g., about 6, 5, 4, 3, 2, 1 hour(s) or less). In some embodiments, expression of the peptide reporter, if present, is detectable in less than 30 minutes (e.g., in about 10, 15, 20, 25. 30 minutes or less).

Any amplification protocol can be used according to the methods provided herein. In some embodiments, amplifying comprises polymerase chain reaction (PCR). This amplification method is convenient if a thermal cycler is available due to ease of readout of particular point mutations. In some embodiments, amplifying comprises isothermal amplification. In some embodiments, the isothermal amplification technique is recombinase polymerase amplification (RPA) or reverse-transcription recombinase polymerase amplification (RT-RPA). Generally, RPA and RT-RPA reactions require near-ambient temperatures (37° C.-42° C.), are completed in fewer than 30 minutes (e.g., about 10, 15, 20, 25, 30 minutes), and yield similar or higher sensitivity as compared to PCR or LAMP techniques, making RPA and RT-RPA methods suitable for portable diagnostics in the field. In some embodiments, RPA is used with the “one-pot” amplification and detection methods provided herein. In such embodiments, the methods comprise performing reverse transcription (RT), RPA, and transcription (TX) methods in a single cell, chamber, reservoir, tube, well, or test tube. In some embodiments, LAMP (loop-mediated isothermal amplification) is performed. In some embodiments, other isothermal amplification methods are used, including: strand displacement amplification (SDA), helicase-dependent amplification (HDA), nicking enzyme amplification reaction (NEAR), signal mediated amplification of RNA technology (SMART), rolling circle amplification (RCA), isothermal multiple displacement amplification (IMDA), single primer isothermal amplification (SPIA), and polymerase spiral reaction (PSR).

Any appropriate sample can be used according to the methods provided herein. In some embodiments, the sample is a biological sample obtained from an individual subject (e.g., a human subject, a non-human mammalian subject) or a collection of subjects. The sample is, in some embodiments, a diagnostic sample. The sample type will vary depending on the target pathogen, target genetic variation, or target nucleic acid. The biological sample can be saliva, a nasopharyngeal swab, blood, serum, sputum, or another matrix. For example, a diagnostic sample for detecting viruses such as SARS-CoV-2 can be a saliva sample, a nasopharyngeal swab sample, a blood sample, or a sputum sample. In some embodiments, samples have been heated prior to performing the other method steps. For instance, samples can be heated to or above a temperature of about 65° C., which kills the virus and releases nucleic acids. In other cases, samples are frozen (e.g., at −80 ° C.) prior to testing. The term “subject” may be used interchangeably with the terms “individual” and “patient” and includes human and non-human subjects. In some embodiments, the subject is a mammal, such as a domesticated or pet mammal.

Other samples appropriate for use according to the methods provided herein can also include, without limitation, food samples, drinking water, environmental samples, and agricultural products. In some cases, samples appropriate for use according to the methods provided herein are “non-biological” in whole or in part. Non-biological samples include, without limitation, plastic and packaging materials, paper, clothing fibers, and metal surfaces. In some embodiments, the methods provided herein are used in food safety and food biosecurity applications, such as screening food products and materials used in food processing or packaging for the presence of pathogens in biological and/or non-biological samples.

In some embodiments, the method is for detecting a pathogen-associated nucleic acid in a biological sample obtained from a subject, where the method comprises: (i) amplifying nucleic acid obtained from the biological sample; (ii) translating the amplified nucleic acid into translation products; and (iii) detecting a translated detectable reporter peptide indicative of the presence of the pathogen-associated nucleic acid in the sample. In some embodiments, any combination of the amplifying, translating, and detecting steps occurs either in a liquid phase or embedded within a porous or paper substrate. In some embodiments, the detecting of a translated detectable reporter comprises detecting a fluorescence or color signal and the fluorescence or color signal is not detectable by the method in the absence of the pathogen-specific target nucleic acid.

As used herein, a pathogen can be any organism, virus, or prion which causes a disease or disorder in a subject, such as a microorganism. A microorganism may be unicellular or multicellular and may refer to a species of bacteria, archaea, or certain eukaryotes, such as microscopic fungi, protists, rotifers, and unicellular plants such as algae.

In some embodiments, the method is used to detect SARS-CoV-2 target sequences in a sample. In some embodiments, the SARS-CoV-2 target sequence is selected from the S, E, N, and ORF1b genes.

In some embodiments, the method is used to detect a frame-altering mutation in a target sequence in a sample. In some embodiments, the method is used to detect a frame-altering mutation in a biological sample derived from a subject, such as, e.g., in a BRCA1 or BRCA2 gene target sequence.

In some embodiments, the method is used to detect a mutation, SNP, or SNV in a target sequence in a sample, such as, e.g., T215F in an HIV reverse transcriptase gene target sequence. In some embodiments, the method is used to detect a mutation, SNP, or SNV in a target sequence in a biological sample derived from a subject, such as, e.g., in a BRCA1, BRCA2, HFE, THMD2, or CTFR gene or gene product. In some embodiments, the method detects the H63D SNP in a human HFE gene target sequence. In some embodiments, the method detects a mutation, SNP, or SNV in a human THMD2 gene target sequence. In some embodiments, the method detects a mutation, SNP, or SNV in a human CTFR gene target sequence.

In some cases, it may be advantageous to adapt the methods described herein for high-throughput, reproducible, and rapid detection, for example, for a clinical setting, at home testing, or in the field.

As used herein, the term “nucleic acid” refers to a polymeric form of nucleotides, which may include both sense and anti-sense strands of RNA, cDNA, genomic DNA, and synthetic forms and mixed polymers thereof. A nucleotide refers to a ribonucleotide, deoxynucleotide or a modified form of either type of nucleotide. The phrase nucleic acid molecule as used herein is synonymous with nucleic acid and polynucleotide. A nucleic acid molecule is usually at least six bases in length, unless otherwise specified. The term includes single- and double-stranded forms. The term includes both linear and circular (plasmid) forms. A polynucleotide may include either or both naturally occurring and modified nucleotides linked together by naturally occurring nucleotide linkages, non-naturally occurring chemical bonds, and/or linkers.

Unless specified otherwise, the left hand end of a polynucleotide sequence written in the sense orientation is the 5′-end and the right hand end of the sequence is the 3′-end. In addition, the left hand direction of a polynucleotide sequence written in the sense orientation is referred to as the 5′-direction, while the right hand direction of the polynucleotide sequence is referred to as the 3′-direction. Further, unless otherwise indicated, each nucleotide sequence is set forth herein as a sequence of deoxyribonucleotides. It is intended, however, that the given sequence be interpreted as would be appropriate to the polynucleotide composition: for example, if the isolated nucleic acid is composed of RNA, the given sequence intends ribonucleotides, with uridine substituted for thymidine.

Nucleic acids and/or other moieties of the invention may be isolated. As used herein, “isolated” means to separate from at least some of the components with which it is usually associated whether it is derived from a naturally occurring source or made synthetically, in whole or in part.

Nucleic acids and/or other moieties of the invention may be purified. As used herein, purified means separate from the majority of other compounds or entities. A compound or moiety may be partially purified or substantially purified. Purity may be denoted by a weight by weight measure and may be determined using a variety of analytical techniques such as but not limited to mass spectrometry, HPLC, etc.

As used herein, the terms “protein” and “polypeptide” are used interchangeably and refer to polymer of amino acid residues, including amino acid polymers in which one or more amino acid residue is an artificial chemical mimetic of a corresponding naturally occurring amino acid, as well as to naturally occurring amino acid polymers and non-naturally occurring amino acid polymer. Multiple polymers of amino acids binding to each other are a protein complex. Protein and polypeptide mean at least two covalently attached amino acids, which includes proteins, polypeptides, oligopeptides and peptides. Methods of manufacturing polypeptides are known to the skilled artisan and further described herein. For example, the polypeptides disclosed herein may be produced in cell-free systems, or in prokaryotic or eukaryotic cells.

### Nucleic Acids and Compositions

In another aspect, provided herein is a composition or primer for use in a method described herein. In some embodiments, the composition is a set of primers representing a primer pair, wherein one primer is a forward primer and the other primer is a reverse primer designed to amplify a nucleic acid sequence of interest. In some embodiments, the primer comprises, consists of, or consists essentially of a nucleic acid described herein.

Provided herein is a nucleic acid or forward primer comprising, from 5′ to 3′, (1) a nucleic acid sequence encoding a ribosome binding site (RBS), (2) a start codon, and (3) a nucleic acid sequence of a first portion of a target nucleic acid sequence. In some embodiments, the nucleic acid or forward primer comprises from 5′ to 3′: (a) (1) a transcriptional promoter, (2) a nucleic acid sequence encoding a ribosome binding site (RBS), (3) a start codon, and (4) a nucleic acid sequence of a first portion of a target nucleic acid sequence; (b) (1) a nucleic acid sequence encoding a ribosome binding site (RBS), (2) a start codon, (3) a nucleic acid sequence encoding a second peptide reporter in-frame with the start codon, and (4) a nucleic acid sequence of a first portion of a target nucleic acid sequence in frame with the second peptide reporter; or (c) (1) a transcriptional promoter, (2) a nucleic acid sequence encoding a ribosome binding site (RBS), (3) a start codon, (4) a nucleic acid sequence encoding a second peptide reporter in-frame with the start codon, and (5) a nucleic acid sequence of a first portion of a target nucleic acid sequence in frame with the second peptide reporter. In some embodiments, the first portion of the target nucleic acid sequence may comprise a coding sequence positioned in-frame with the start codon.

Provided herein is a nucleic acid or reverse primer comprising, from 5′ to 3′, (1) a reverse complement of a stop codon in-frame with (2) the reverse complement of the sequence encoding a first peptide reporter in frame with (3) a nucleic acid sequence complementary to a second portion of the target nucleic acid sequence. The reverse primer is designed such that the second portion of the target nucleic acid sequence comprises a coding sequence positioned in-frame with the sequence encoding the peptide reporter in the amplified product. In some embodiments, the second portion of the target nucleic acid sequence when amplified will include a protein coding sequence in-frame with a start codon within the target nucleic acid sequence.

The skilled worker will appreciate that while the relative position of the features of each nucleic acid is set as described above, e.g., features (1), (2), and (3) and sometimes (4) and/or (5), the composition of each feature and the spacing between features can vary without affecting a primer function. For example, a forward primer disclosed here may comprise three to five specific features: a (1) RBS, (2) start codon, and (3) portion of a target sequence, and optionally a (4) transcriptional promoter and/or (5) nucleic acid encoding a second peptide promoter. Various sequences may be used to provide these features to a nucleic acid or forward primer. The skilled worker will appreciate that various sequences encoding peptide reporters may be suitable such that the peptide report can be incorporated into a translated product of a translation reaction and then detected in a detection step indicating the presence of a target nucleic acid in a sample. The skilled worker will appreciate that various RBS's may be suitable to provide docking of a ribosome in translation reaction, for e.g., various prokaryotic or eukaryotic RBS's depending on the reaction being used. The skilled worker will appreciate that depending on the system being used, multiple start codon sequences may be suitable, such as, different prokaryotic start codons. The skilled worker will appreciate that various stop codon sequences are suitable for terminating translation of a molecule in a translation reaction, for e.g., various prokaryotic or eukaryotic stop codons depending on the translation reaction. The skilled worker will appreciate that various transcriptional promoters are suitable for providing transcription of an amplified nucleic acid in a transcription reaction, for e.g., various prokaryotic or eukaryotic promoters depending on the transcription reaction.

The skilled worker will appreciate that, in particular, the target nucleic acid sequence should be tailored to the particular application for which the primer will be applied to. As described herein, various target sequences may be chosen, such as, e.g., from a pathogen or genetic marker. In some cases, the target sequence may have a mutation, e.g. a SNV, point mutation, or frame-altering mutation. In some cases, the target sequence may be specific to a specific pathogen variant or genetic marker. The skilled worker will appreciate that there exists almost limitless variations in the target sequence which may be used, which may, in some cases depend on the type of sample used and the parameters of the amplification step and/or the detection step. In some cases, it may be advantageous to adapt the primers described herein for high-throughput, reproducible, and rapid detection, for example, for methods of nucleic acid detection in a clinical setting, in at-home setting, or in the field.

Provided herein is a composition comprising: (a) a forward primer comprising, from 5′ to 3′, a nucleic acid sequence encoding a ribosome binding site (RBS), a start codon, and a nucleic acid sequence of a first portion of a target nucleic acid sequence; and/or (b) a reverse primer comprising, from 5′ to 3′, a reverse complement of a stop codon in-frame with a reverse complement of a sequence encoding a first peptide reporter a nucleic acid sequence and a reverse complement of a second portion of the target nucleic acid sequence. In some embodiments, the composition comprises a forward primer comprising from 5′ to 3′: (a) a transcriptional promoter, a nucleic acid sequence encoding a ribosome binding site (RBS), a start codon, and a nucleic acid sequence of a first portion of a target nucleic acid sequence; (b) a nucleic acid sequence encoding a ribosome binding site (RBS), a start codon, a nucleic acid sequence encoding a second peptide reporter in-frame with the start codon, and a nucleic acid sequence of a first portion of a target nucleic acid sequence; or (c) a transcriptional promoter, a nucleic acid sequence encoding a ribosome binding site (RBS), a start codon, a nucleic acid sequence encoding a second peptide reporter in-frame with the start codon, and a nucleic acid sequence of a first portion of a target nucleic acid sequence.

The skilled worker will appreciate that variations of any of the primers mentioned herein may be used as a nucleic acid of the disclosure or in a composition of the disclosure. Provided herein is a composition comprising a forward primer and a reverse primer, wherein the respective forward and reverse primers comprises, consists essentially of, or consists of a nucleotide sequence having at least 80%, 85%, 90%, 93%, 95%, 96%, 97%, 98%, 99%, or 99.5% identity respectively to SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, and SEQ ID NO: 59 and SEQ ID NO: 60. In some embodiments, the forward and reverse primers comprise, consist essentially of, or consist of a nucleotide sequence selected from SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, and SEQ ID NO: 59 and SEQ ID NO: 60.

Provided herein is a nucleic acid or primer comprising, consisting of, or consisting essentially of a nucleic acid having at least 80%, 85%, 90%, 95%, 96%, 97%, 98%, or 99% identity to any one of SEQ ID NOs: 1-60.

The skilled worker will appreciate that variations of any of the primers described herein may be used as a primer in a method or composition of the disclosure, such as wherein the target sequence is altered but the other features are maintained (e.g. a RBS, a start codon, a sequence encoding a peptide reporter or complement thereof, and/or a reverse complement of a stop codon 5′ of and in-frame with a reverse complement of a sequence encoding a peptide reporter). Provided herein is a nucleic acid or primer comprising, consisting of, or consisting essentially of a nucleic acid having at least 90%, 95%, 96%, 97%, 98%, or 99% identity to any one of SEQ ID NOs: 1-60 outside of the nucleic acid sequence of a portion of the target sequence (or a complement thereof for a reverse primer), but wherein the portion of the target sequence, or complement thereof, has zero sequence identity or less than 1%, 3%, 5%, 10%, 20%, or 40% sequence identity to any one of SEQ ID NOs: 1-60 for just that sub-region.

“Percent (%) identity” with respect to a nucleic acid sequence is defined as the percentage of nucleotides in a candidate sequence that are identical with the nucleotides in the specific sequence, after aligning the sequences and introducing gaps, if necessary, to achieve the maximum percent sequence identity. The degree of identity between a nucleotide sequence of the disclosure (“disclosure sequence”) and the nucleotide sequence may be calculated as the number of exact matches in an alignment of the two sequences, divided by the length of the “disclosure sequence,” or the length of the specific nucleotide sequence, whichever is the shortest. The result is expressed in percent identity. Alignment for purposes of determining percent sequence identity can be achieved in various ways that are within the skill in the art, for instance, using publicly available computer software such as BLAST, BLAST-2, ALIGN, ALIGN-2, or Megalign (DNASTAR) software. Those skilled in the art can determine appropriate parameters for measuring alignment, including any algorithms needed to achieve maximal alignment over the full-length of the sequences being compared.

### Articles of Manufacture

In another aspect, the present invention provides articles of manufacture useful for detecting a target nucleic acid in a sample according to the methods provided herein, such as a device or kit.

In some embodiments, the article of manufacture is a kit for detecting a target nucleic acid in a sample comprising a first peptide primer and/or second peptide primer, and another agent or device; wherein at least one peptide primer comprises a sequence encoding a peptide reporter or a complement thereof. The kit may comprise: (a) a forward and reverse primer composition described herein, and (b) a reagent or device. In some embodiments, the reagent is: an agent capable of binding the first or second peptide reporter, an agent capable of binding the first or second peptide reporter to form a detectable agent, a detectable agent which binds the first or second peptide reporter; or a paper test article. In some embodiments, the kit is for detecting a target nucleic acid of a pathogen (e.g. of a specific strain or variant of a pathogen), such as a virus or bacterium for diagnostic purposes. In other embodiments, the kit is for detecting a target nucleic acid in a biological sample from a subject for genetic diagnostic purposes such as, for example, identifying the presence of a disease-causing or disease-predisposing single nucleotide polymorphism or other genetic mutation. Optionally, the kit can include instructions and/or additional reagents or devices for performing a detection method provided herein.

In some embodiments, the peptide primer comprises a nucleic acid complementary to a portion of a target sequence comprising a SNP or SNV. In some embodiments, the agent is a component for use in an amplification or translation reaction, such as, e.g., a component of a PCR, RPA, RT-RPA, or cell-free translation or cell-free transcription-translation reaction. In some embodiments, the agent binds to the peptide reporter and/or is a detectable agent, such as, for example, an antibody or a fluorescent, luminescent, or colorimetric agent (e.g. a GFP1-10, sfCherry1-10, or lacZ-ω protein). In some embodiments, the kit comprises a preserved paper test article, such as a freeze dried paper article.

In some embodiments, the kit comprises a device for use in cell-free translation reaction or cell-free transcription-translation reaction, such as a device described herein. In some cases, the device comprises a preserved paper test article, upon which any step(s) of the methods provided herein can be performed. In some embodiments, the paper test article is preserved by freeze-drying. In some cases, the device comprises a test tube, upon which any step(s) of the methods provided herein can be performed. For example, nucleic acids encoding the peptide reporter primers and other reaction reagents can be freeze-dried in test tubes to render them stable at room temperature. Freeze-dried components can be reactivated upon addition of a sample and water, and can report on the presence of an endogenous nucleic acid of interest in the sample. In such cases, the peptide reporter primers and methods provided herein can be performed at a cost of less than $3 per assay.

In some cases, the device is used with a portable electronic reader. In this manner, the electronic reader serves as companion technology that provides robust and quantitative measurements of device outputs. In some embodiments, the electronic reader comprises readily available consumer components, open-source code, and laser-cut acrylic housing, and is powered by a rechargeable lithium ion battery. The electronic reader can further comprise an onboard data storage unit. In some cases, to achieve sensitive detection of toehold switch signal output, an acrylic chip that holds the freeze-dried, paper-based reactions is placed into the reader between a light source (e.g., to read optical density at excitation and emission wavelengths of light appropriate for and characteristic of a particular detectable reporter) and electronic sensors. In some cases, the light source is a light emitting diode (LED) light source. Samples can be read using onboard electronics. In this manner, a portable electronic reader can provide low-noise measurements of changes associated with the reporter element including changes in fluorescence transmission.

In some embodiments, the article of manufacture is a device, such as, a device described above or a device adapted for molecule detection. In some embodiments, the device adapted for molecule detection, such as for detecting a peptide reporter or performing one or more steps of a method described herein (e.g. amplifying, translating, and/or detecting). In some embodiments, the device is portable and/or configured to be used in the field, at point of care (e.g. in a clinic), or by a consumer in an at-home setting. In some embodiments, the device is for detecting a target nucleic acid of a pathogen (e.g. of a specific strain or variant of a pathogen), such as a virus or bacterium for diagnostic purposes. In other embodiments, the device is for detecting a target nucleic acid in a biological sample from a subject for genetic diagnostic purposes such as, for example, identifying the presence of a disease-causing or disease-predisposing single nucleotide polymorphism or other genetic marker or mutation.

In some embodiments, the device is adapted to perform a PCR or isothermal amplification suitable for use a method described herein. In some embodiments, the device is adapted to perform a cell-free translation and/or cell-free transcription-translation reaction, such as by comprising one or more chambers or reservoirs for performing an individual reaction in. In some embodiments, the device comprises a preserved paper test article, such as a freeze dried paper article, upon which one or more steps of a method described herein may be performed. The device can be pre-loaded with one or more freeze-dried reagents or reaction mixtures.

In some embodiments, the device comprises an electronic display and/or user interface. In some embodiments, the device comprises software, firmware and/or hardware, such as a battery, data storage unit, light source, and/or sensor. In some embodiments, the sensor is a light and/or color sensor and reporter signals from performing a method disclosed herein are detectable, including quantitatively and with low-noise measurements. In some embodiments, the device is capable of emitting a signal, visible to the naked eye, caused by the detecting step and indicating the presence of a target nucleic acid in the sample as a result of performing a method described herein, such as a fluorescent or colorimetric signal. In some embodiments, the device does not comprise any thermal cycling equipment.

In some cases, it may be advantageous to adapt the kits and devices described herein for high-throughput, reproducible, and rapid detection, for example, in a clinical setting, in at-home setting, or in the field. In some cases, peptide reporter primers of this disclosure, reagents for performing isothermal amplification, and reagents for cell-free transcription-translation reactions can be provided in a device configured for rapid, reproducible detection in a clinical setting.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

The present embodiments have been described in terms of one or more embodiments, and it should be appreciated that many equivalents, alternatives, variations, and modifications, aside from those expressly stated, are possible and within the scope of the claims.

### EXAMPLES

The examples below demonstrate a rapid and low-cost platform for nucleic acid detection that employs the translation process along with peptide reporters to yield convenient portable assays. These assays can provide clear colorimetric or fluorometric results readily discerned by eye and can obviate the need for thermal cycling equipment by employing isothermal reactions. The peptide reporters are appended to the targeted nucleic acid sequence by extended primers used during an amplification step. The identity of the amplified nucleic acid sequence can then be verified using cell-free transcription-translation reactions directly to provide signal outputs. Here, it is demonstrated that this nucleic acid detection platform can be applied to pathogen detection by applying it to SARS-CoV-2. Furthermore, it is demonstrated that this platform can be employed to detect subtle genetic changes like single nucleotide polymorphism and frame-altering mutations associated with cancer and inherited disorders. Finally, this section demonstrated that the platform is compatible with recombinase polymerase amplification and paper-based cell-free reactions to provide directly visible colorimetric results to provide point-of-care genetic testing and virus detection. This platform can provide individuals with easier and more private access to genetic information that can give genetic susceptibility, disease and treatment guidance.

### Example 1

### De novo Peptide Primer Design Strategy

To develop a portable assay that directly took advantage of sequence-checking capacity of translation, the inventors began to investigate split fluorescent reporter protein systems developed for live-cell imaging. These systems divide fluorescent reporters into individual fragments based on their structure. On their own these fragments are non-fluorescent. When both are present, they spontaneously self-assemble to produce a detectable fluorescent signal. One fragment in the split pair is often relatively short (˜16 residues), to facilitate tag addition for in vivo fluorescent imaging studies. In many situations, the splitting point in the parent reporter protein is located in a loop region between two well-defined secondary structure motifs. For example, by isolating the eleventh β-strand, GFP can be split into two fragments: GFP11 and GFP1-10, with GFP11 being a short, 16-amino-acid peptide. Importantly, the short length of GFP11 and other split reporter proteins may allow for incorporation of the peptide sequence into amplification primers to provide a translation-based readout for nucleic acid testing.

To exploit such peptide reporters, primers with flanking regions that append transcriptional and translational signals into the resulting amplicon were designed. To do this, both forward and reverse primers have binding regions complementary to the target sequence and 5′ regions encoding the necessary output signals (FIG. 1A). A typical signal section in the forward primers contain a T7 promoter sequence, followed by a prokaryotic ribosome binding site (RBS) and a start codon sequence. The signal section of a reverse primer contains the reverse complement of the peptide reporter followed by an in-frame stop codon. These transcriptional and translational regulatory elements thus enable the amplified target DNA to activate transcription and translation in the cell-free system. GFP11, serving as a reporter, will produce an output if the upstream signal elements and the amplified fragment can be translated. sfCherry11 and lacZ-α peptide have been successfully used as reporters to provide fluorescence and colorimetric readout options, but this reporter in principle can be any peptide with reporting function that can be conveniently incorporated into an amplification primer. Currently, oligonucleotides up to 200 bases can be readily synthesized, providing peptides of up to about 59 residues to be used. In principle, these translation-based assays allow for detection through at least two different modalities: (1) confirmation of the presence of an amplified product with translation verifying that the sequence encodes a functional open reading frame, and (2) distinguishing different amplified products based on the presence of an open reading frame (FIG. 1B). The latter detection modality can be broadly used to identify both frame-shift mutations and nonsense mutations that generate premature stop codons. To design priming sequences, general primer design strategies for PCR and recombinase polymerase amplification (RPA) were used. The inventors further developed an automated design code to minimize primer secondary structure and optimize translational output. FIG. 1C depicts the general workflow of the platform. First, primers targeting specific DNA/RNA are in silico designed. The samples obtaining from individuals are subjected to lysis and extraction. The extracted DNA/RNA can then be added to the amplification mix. Amplification products are added directly to the cell-free reaction. Fluorescence/colorimetric signals can then be observed, measured, and/or recorded.

### Pathogen and SNP Detection Using Peptide Reporter Primer Platform

The inventors tested primers appending the GFP11 peptide reporter to a nucleic acid amplification product. The system was expected to produce fluorescence signal only when the target sequence is present and amplified. To respond to the COVID pandemic, the inventors designed peptide reporter primers targeting SARS-CoV-2. In the presence of SARS-CoV-2 RNA, the primers were able to amplify the RNA and expressed GFP11, which reassembles with the GFP1-10 provided in the cell-free system, resulting in green fluorescence (FIG. 2C). These results demonstrated that the SARS-CoV-2 E and N genes could be detected with this GFP peptide reporter system using synthetic SARS-CoV-2 RNA (FIG. 2D-G). Synthetic SARS-CoV-2 RNA is added to the amplification mix along with primers designed to target the envelope (E) gene, nucleoprotein (N) gene, or the ORF1b region of the virus (FIG. 2E). Positive samples will allow for nucleic acid amplification and production of GFP11 peptides, which will self-assemble with the GFP1-10 protein supplied in the cell-free reaction to produce green fluorescence. The results indicate that this platform can be used to detect pathogens through nucleic acid presence.

To identify another method of detecting nucleic acids using peptide reporters, the 3′ nucleotide mismatch in the primers for the recombinase polymerase amplification (RPA) was exploited for its impact on amplification efficiency. The amplification can be prevented by the presence of 3′ mismatches. To test if this also applies to the peptide reporter primers, primers with 3′ single nucleotide mismatches were designed and tested to detect single nucleotide variants (SNV). The priming sequence of the primers has an exact match to the wild type sequence with a 3′ single nucleotide mismatch to the SNP variant, resulting in fluorescence in only the wild type sequence (FIG. 3A). H63D, a common pathogenic point mutation in HFE gene related to hemochromatosis, was tested. Fluorescence was only observed in the wild-type sample but not the H63D variant, suggesting the primers only amplify the wild type (FIG. 3B). A similar result was also observed for T215F (FIG. 3C), an HIV drug resistance mutation.

These results together indicate that the peptide reporter primers can be used to detect presence of nucleic acids and have single nucleotide specificity through rational design of primers and the sequence-reading properties of translation.

Detection of Frame-Altering Mutations and the Implementation of a Self-Calibrating Dual Fluorescence System

Nonsense mutations and frameshift mutations are two types of mutations that cause large-scale changes to proteins by causing premature termination. Because both mutations alter the wild-type open reading frame, these two types of mutations are referred to herein as frame-altering mutations (FIG. 3B). The main idea of this platform is to take advantage of changes to translation to provide different outputs, thereby detecting these types of mutations.

To do this, primers were designed to amplify the mutation region of interest. Using this set of primers, both the wild-type sequence and the mutant sequence can be amplified, yet the downstream reporter gene is in the same reading frame with the wild-type gene. Therefore, in the subsequent translation reaction, the reporter gene will be expressed to provide readout. However, if there are any frame-altering mutations, the production of the reporter peptide will be prevented because of the premature stop codon due to either a nonsense mutation or the shift of the open reading frame (FIG. 4A).

Using mutations from breast cancer gene 1 (BRCA1) and breast cancer gene 2 (BRCA2) as examples (FIG. 4C-F), it was determined that the reporter platform can be used to detect frame-altering mutations. All four samples provided significantly higher GFP fluorescence for the wild-type samples compared to the samples containing BRCA1/BRCA2 mutations. The detection strategy was applied to eight different BRCA1/BRCA2 mutations and demonstrated clear differences in signal between the wild-type and mutant samples (FIG. 4G). FIG. 4H shows a photograph of the fluorescence obtained from the liquid-phase cell-free reactions. The fluorescence output from the reactions is sufficiently strong to be able to be detected using a smartphone camera using blue-light illumination and an optical filter to exclude the excitation wavelengths.

To provide a more quantitative detection of the mutations, a second short fluorescent tag, sfCherry₂11, was attached to the forward primers (FIG. 5A), which may present a fluorescent signal independent of the amplified open-reading frame to provide a system self-calibration (FIG. 5B). FIG. 5C shows that the red fluorescence for wild type and mutant sample are essentially the same, indicating that the difference observed in green fluorescence between wild type and mutant is because of the presence of mutations rather than differences in amplification yield (FIG. 5D). This capability can be very useful when the actual mutation is heterozygous. By calculating the ratio of green fluorescence to red fluorescence, it is possible to tell whether the sample tested is wild type or mutant and whether it is homozygous or heterozygous. This is also a fail-safe measure to prevent human error (e.g., pipetting mistake) from affecting the results. The red fluorescence signal can be used to normalize to ensure accurate detection. This dual fluorescent readout detection system offers a semi-quantitative method for identifying frame-altering mutations.

The frame-altering and dual output strategies can also be applied to discriminating closely related target sequences, such as, related target sequences of different genetic markers or different pathogen species, subtypes, or variants. For instance, the frame-altering and dual output strategies can be applied to discriminating closely related pathogen strains. For example, SARS-CoV-2 has significant sequence homology with SARS-CoV, the causative agent of the 2003 SARS outbreak. To implement such assays for SARS-CoV-2, multiple regions were identified in the antisense orientation of the N gene that had corresponding stop codons in the N gene of SARS-CoV and designed amplification primers with sfCherry₂11 and GFP11 output signals (FIG. 6A). The resulting assays successfully discriminated between SARS-CoV-2 and SARS-CoV, generating stronger GFP fluorescence after the calibrating sfCherry red fluorescence was taken into account (FIG. 6B-E).

### Rapid, Low-Cost snd Colorimetric Detection in Paper-Based Platforms

FIG. 7A shows that the peptide reporter primers can couple with RPA to detect nucleic acids in clinical serum samples. Experiments were conducted to detect frameshift mutations on BRCA1 or BRCA2 genes using the methods of this disclosure. For different types of BRCA1 or BRCA2 mutations (FIG. 7B-C), the fluorescence of wild type samples is higher than mutant samples, suggesting the presence of mutations. Notably, the fluorescence signal for clinical mutant samples is higher than the synthetic samples tested. This is because the BRCA mutations in humans are usually heterozygous, meaning only one allele is mutated. Homozygous mutants on BRCA1/BRCA2 have lethal effects. The limit of detection for the test was 80 aM of BRCA1 nucleic acid in the amplification reaction and about 600 aM in the starting sample (FIG. 7D-E).

While it can detect presence of nucleic acid and mutations, fluorescent reporters require additional instrumentation to detect. Paper-based cell-free reactions, alternatively, can provide reduce assay costs by using smaller reaction volumes and facilitate distribution for potential point-of-care application. The lacZ gene which encodes β-galactosidase has been widely used as colorimetric reporters and has been shown to work on the paper platform. Because the full length lacZ gene is approximately 3 kb, primers using the 39-residue lacZ-α peptide as the reporter peptide was explored to provide a colorimetric readout. Upon production, the lacZ-α peptides will reassemble with lacZ-α protein supplied in the cell-free system to form functional β-galactosidase, which catalyzes a colorimetric reaction, cleavage of the initially yellow substrate chlorophenol red-β-D-galactopyranoside to purple (FIG. 7F). With the correct production of the lacZ-α, the wild-type samples displayed a color change from yellow to purple, while the mutant samples will remain yellow (FIG. 7G-H). By using the lacZ-α peptide reporter, the readout can be colorimetric, and the detection can be paper-based. This will further provide easy reading of the results, and the nucleic acid detection test can be deployable and suitable for point-of-care use.

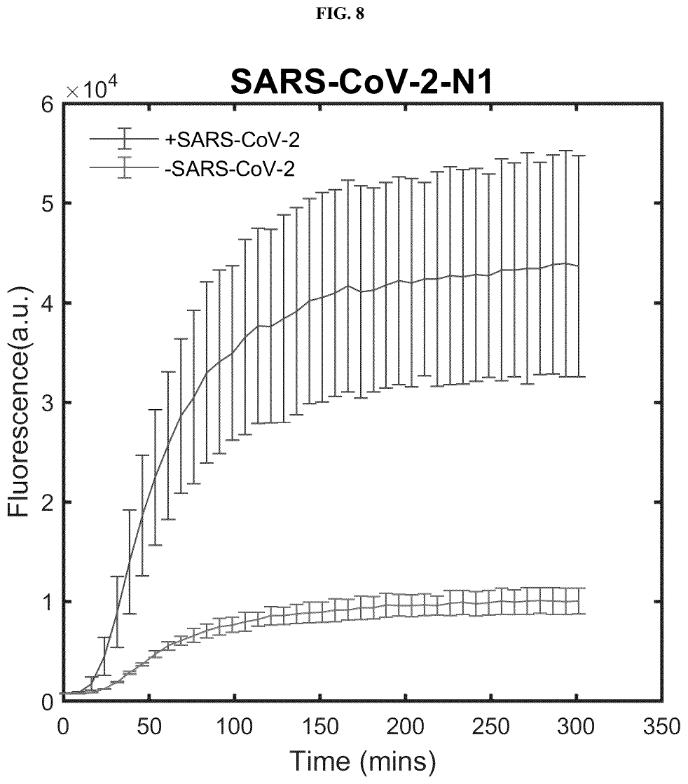

A two-step amplification reaction was also investigated to lower assay detection limits, which can be advantageous for viral testing. SARS-CoV-2 viral RNA was first amplified using a pair of short RPA primers targeting the N gene of the virus (forward: 5′-ACGAACAAACTAAAATGTCTGATAATGGAC-3′ (SEQ ID NO:71), reverse: 5′-GTTTTGATCGCGCCCCACTGCGTTCTCCAT-3′ (SEQ ID NO:72)). These primers span a region extending beyond the binding sites of the forward and reverse peptide primers enabling the latter primers to be used in a subsequent amplification. The first amplification reaction was carried out at 37° C. for 20 minutes. The reaction products were then transferred into a 20-minute RPA reaction using the peptide primers. The products of the second RPA step were then added to liquid-phase cell-free transcription-translation reactions and monitored for fluorescence using the GFP11/GFP1-10 system. FIG. 8 shows time-course measurements of sensor fluorescence demonstrating a significant increase in output for assays supplied with 50 aM of SARS-CoV-2 RNA. This 50 aM assay detection limit is improved to lower concentration detection limits using better-optimized primers for the initial RT-RPA reaction.

### Example 2

### TRANslation of Sequence Labelled TRanscripts (TRANSLATR)

### sfCherry11 Tag as an Alternative Output

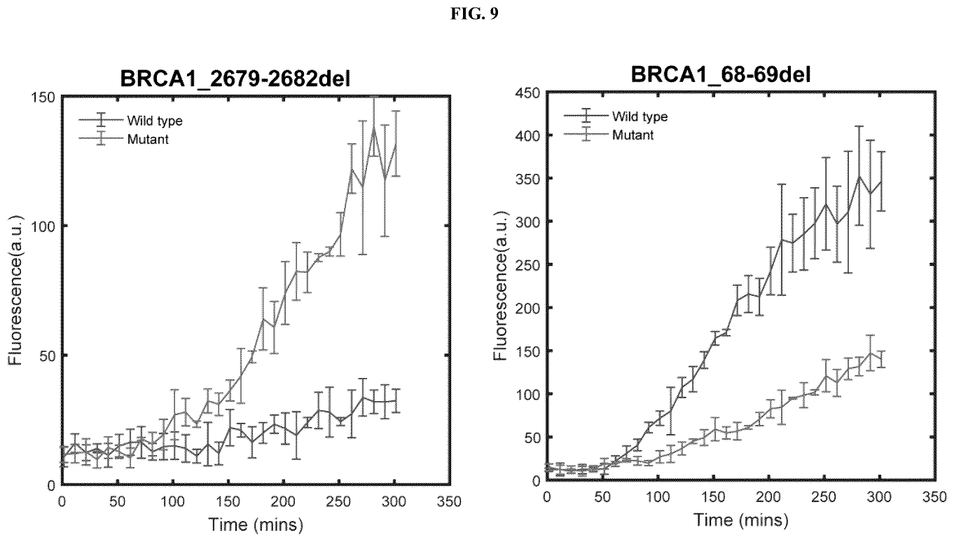

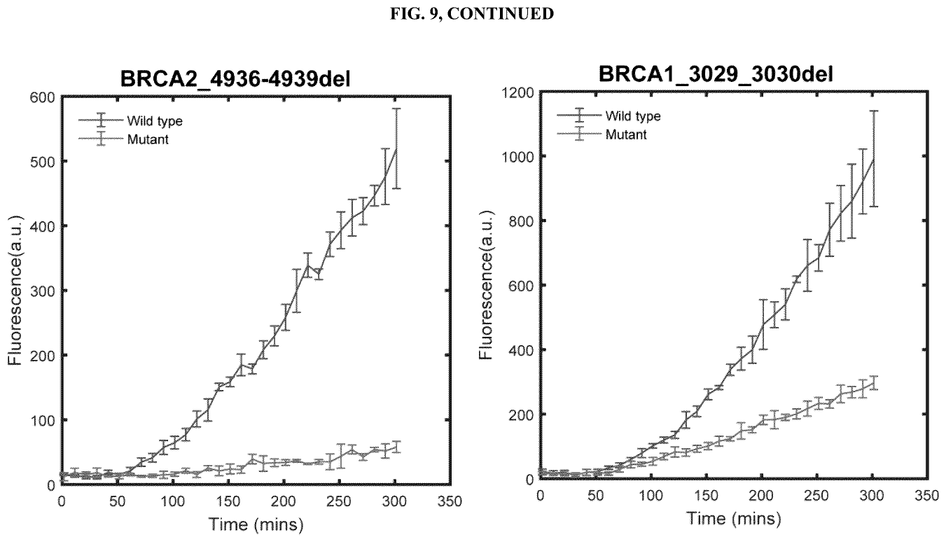

To increase the multiplexing potential of the assay, tandem copies of the sfCherry11 peptide were incorporated into the reverse amplification primers to generate a strong red fluorescence signal after translation. FIG. 9 shows the results from these experiments when tested using BRCA1/BRCA2 cDNA. All four sets of primers were used to amplify the 2679_2682del, 3029_3030del, 68_69del of the BRCA1 and 4936_4939del of BRCA2 genome cDNA in PCR reactions and were then translated in cell-free reactions. All four primer pairs showed clear differences in red fluorescence between the wild-type samples and the mutant samples, which contain stop codons to prevent translation of the dual sfCherry output peptides. This alternative fluorescent peptide tag can thus be used for multiplexed detection reactions in tandem with GFP11, or as a calibration standard when used in the forward primer.

### Demonstration of the Assay for Additional Mutations

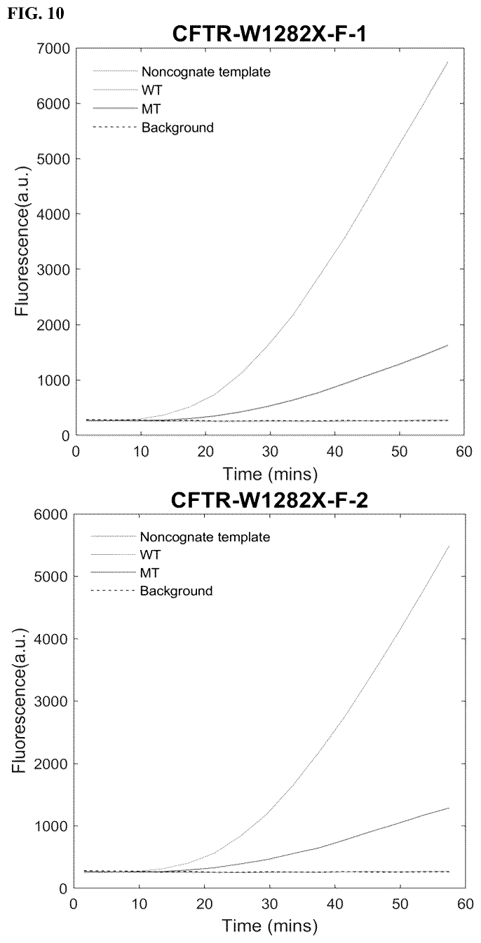

Using the GFP11 as the reporter, an assay was developed to detect the CFTR W1282X mutation. This mutation creates a stop codon that prevents translation of the complete CFTR protein and leads to cystic fibrosis. Fluorescence output from the experiment is shown in FIG. 10. Strong fluorescence produced by reconstitution of GFP11 and GFP1-10 indicates the presence of the wild-type DNA, while low fluorescence indicates the CFTR W1282X mutation.

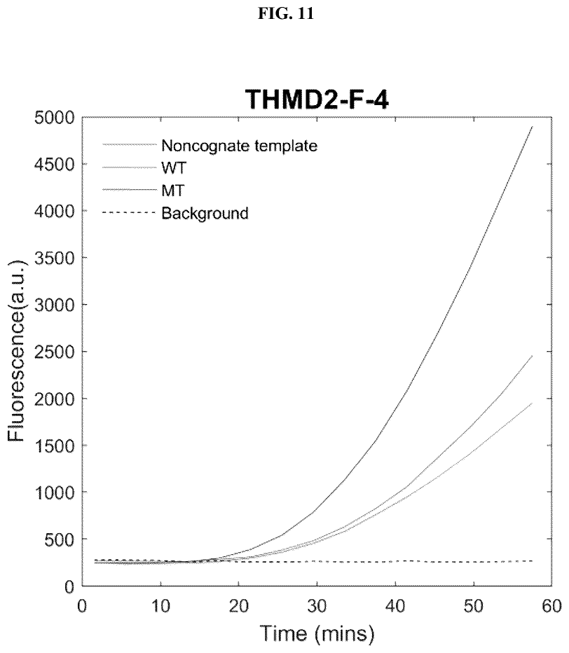

An assay for detection of Thiamine Metabolism Dysfunction syndrome 2 (THMD2) was developed to demonstrate the utility of this technology for rapid genetic testing. THMD2 is an autosomal recessive disorder that leads to impaired delivery of thiamine to the brain (Owen, M., et al., N Engl J Med. 2021; 384: 2159-61). The disorder causes infantile encephalopathy but can be treated through administration of thiamine and biotin. The condition is quite rare and challenging to diagnose since there are over 1000 genetic disorders known to cause infantile encephalopathy. The detection assay was implemented using primers designed to generate GFP11. After a brief RPA step for 8 minutes at 42° C., the resulting amplicons were read using cell-free reactions. The translation step produced a clearly detectable green fluorescence signal within 30 minutes (FIG. 11), leading to a combined assay time of 38 minutes. Such rapid assays can be used to detect a variety of genetic disorders in infants, or other subjects, in a timely fashion.

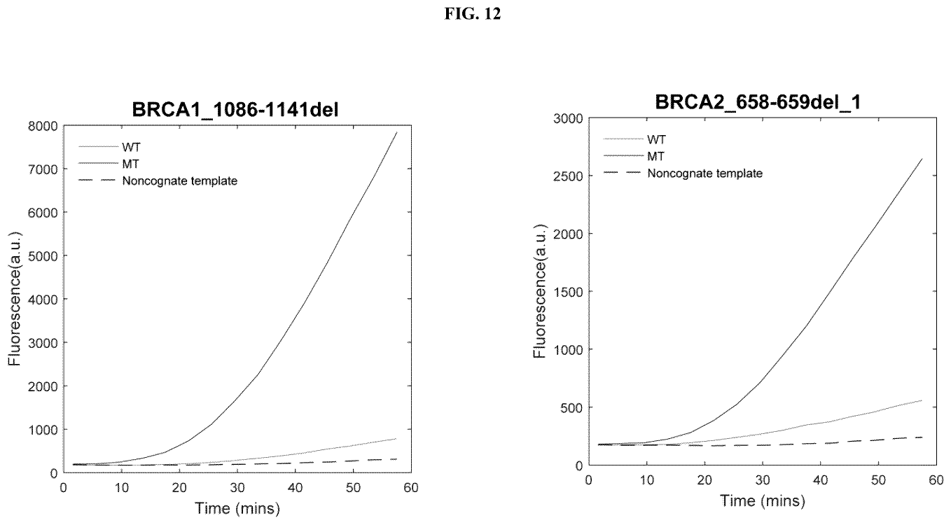

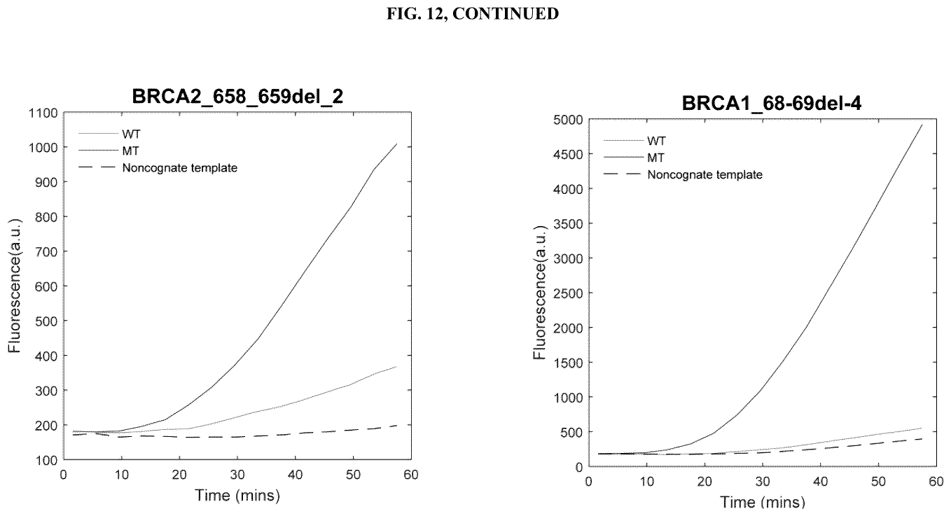

Translation-based assays to detect BRCA1/BRCA2 mutations from genomic DNA were also developed. Use of genomic DNA, which can include introns not present in cDNA or afford sequences that are translated in the antisense orientation, provided for the development of assays that produce a fluorescence output in the presence of the mutation of interest, rather than the wild-type sequence. FIG. 12 displays the fluorescence time-course measurements for four different mutations. All four assays showed strong fluorescence in the presence of the mutations and significantly lower fluorescence against the wild-type and noncognate template.

### Detection of BRCA1/BRCA2 Mutations from cDNA With Self-Calibrated Reporting

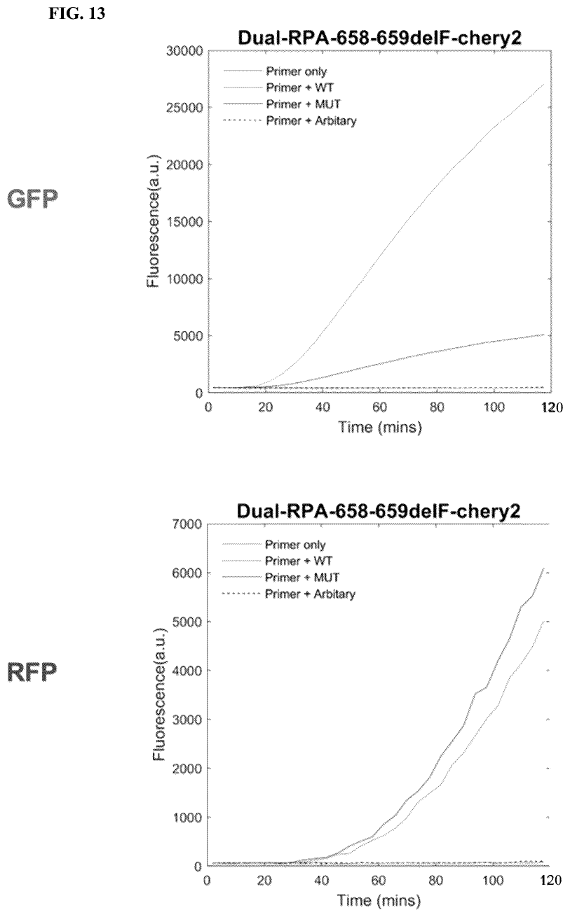

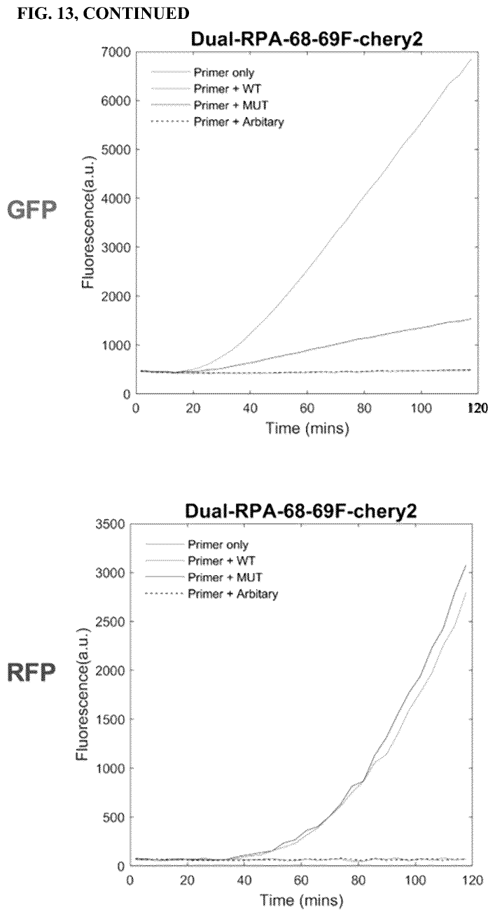

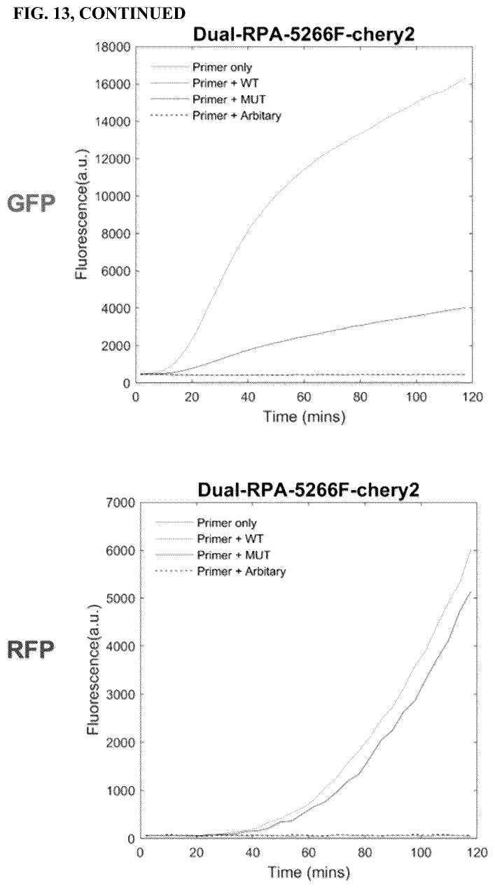

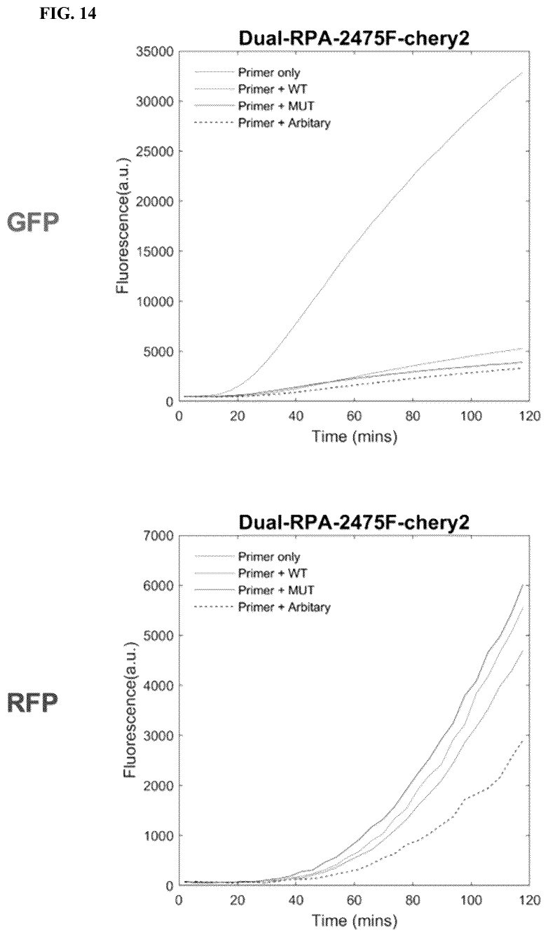

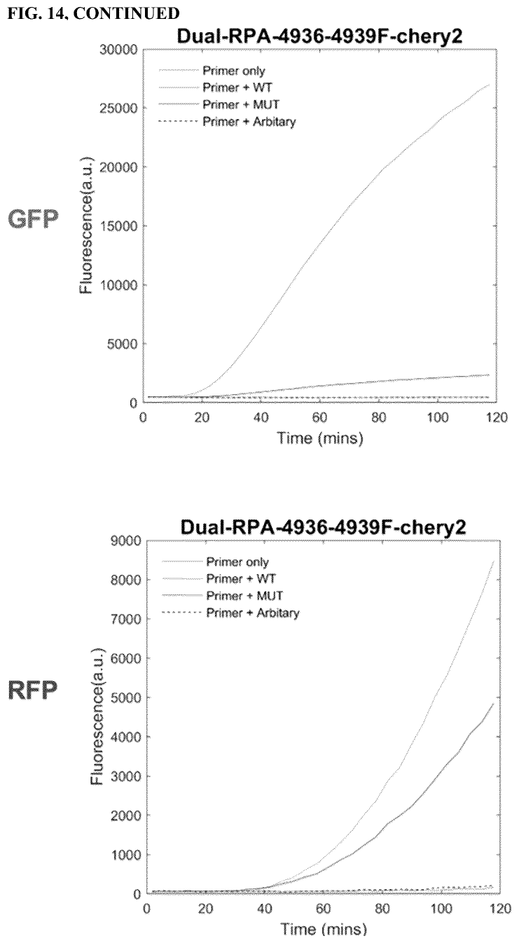

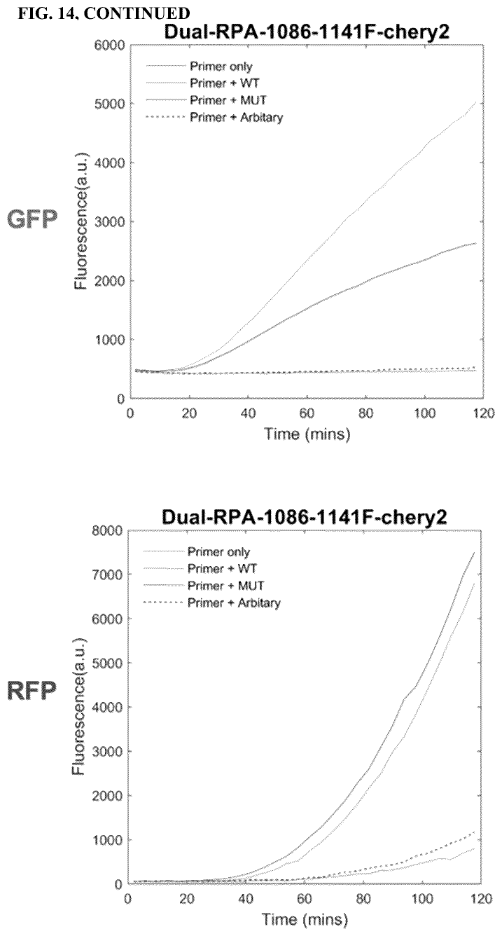

In some cases, the amplification step by PCR or RPA or RT-RPA in the assay may produce varying amounts of DNA as a result of sample-to-sample variations, low template concentration, or other effects. In these cases, it is valuable to have an internal control signal that provides a measure of the amount of DNA generated from the amplification reaction. This self-calibration was accomplished by adding the sfCherry2 peptide into forward primer, thus ensuring that red fluorescence would be generated for each expression cassette generated by the forward primer. The signal for translation-based readout was the GFP11 peptide incorporated into the reverse primer. FIG. 13 and FIG. 14 show the output of these primer systems against mutations 5266dup, 68_69del, 2475del and 1086_1141del in the cDNA of the BRCA1 gene and 658_659del and 4936_4939del in the cDNA of the BRCA2 gene. Much stronger GFP output was observed for the wild-type samples compared to the mutant samples. In addition, significant RFP (red fluorescent protein) output was observed across all the samples and provides a valuable calibration signal. In samples where only primers were added or there was a non-cognate template present, RFP output was either completely abolished (FIG. 13) or considerably smaller (FIG. 14) than that observed for the wild-type and mutant samples.

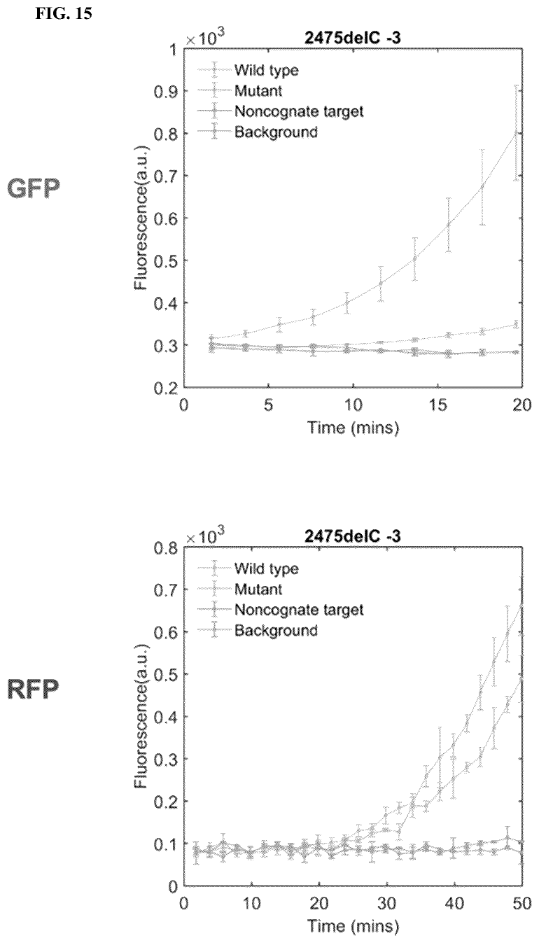

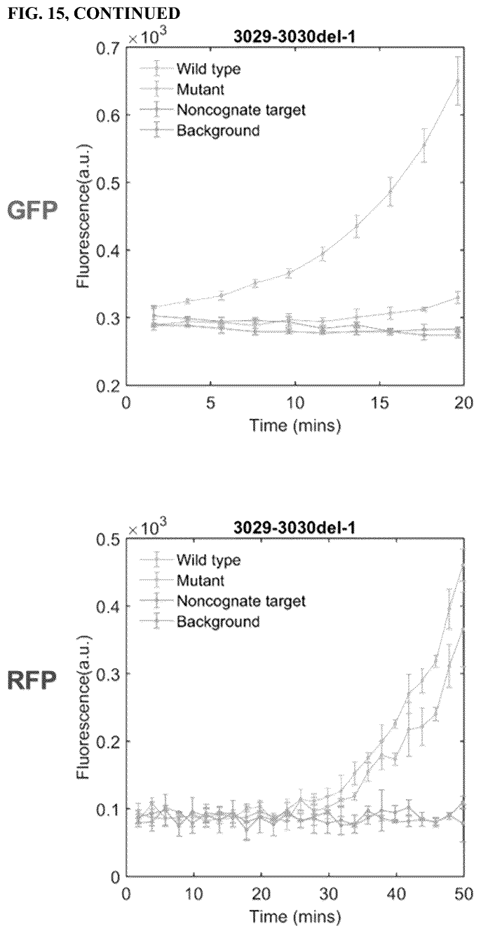

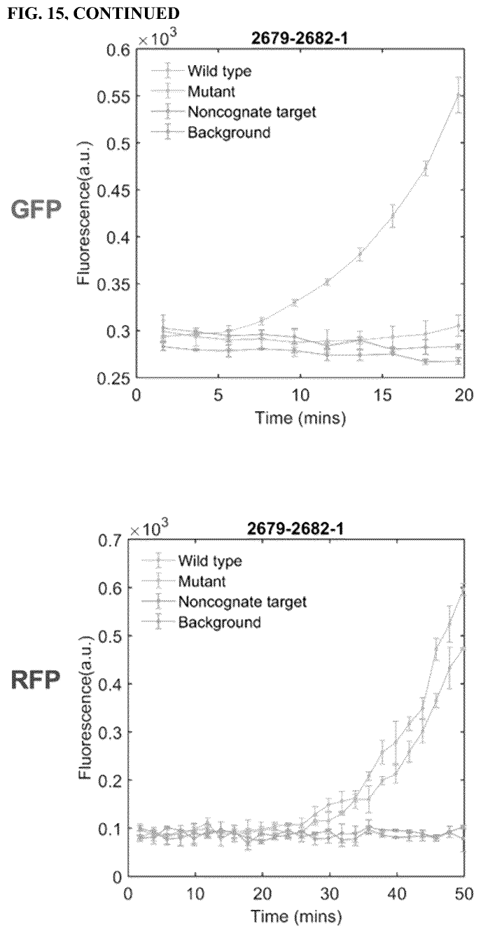

The inventors performed additional sequence optimization experiments and developed enhanced self-calibrating dual-channel fluorescence primers for detection of 2475del, 3029_3030del and 2679_2682del in the cDNA of the BRCA1 gene. As shown in FIG. 15, these primers showed extremely low levels of RFP fluorescence for the samples not expected to generate any amplicon products (noncognate target). Furthermore, these primers showed strong fluorescence for detection of the wild-type or mutant depending on the particular mutation and primers used, thus providing rapid identification of the BRCA1/BRCA2 mutations.

As demonstrated herein, a rapid, low-cost detection method has been developed for detecting frameshift and nonsense mutations and for pathogen identification using an isothermal, in vitro translation-based assay. Compared with conventional assays, the isothermal, in vitro translation-based assay has several desirable features. Reactions can be accomplished in about 1 hour, depending on the sample concentration and cell-free reaction speed. It uses translation-based sequence confirmation, which can be specific down to the single-nucleotide level for any mutation that produces a stop codon and does not require deep knowledge of nucleic acid interactions. It provides visible readouts that can be read directly by eye or with simple equipment, facilitating use at both point of care and in-home settings. Lastly, the assay is inexpensive and temperature stable, costing only about $3 per test and using chemical components that are readily lyophilized.

TABLE 1
Primer sequences used for SARS-COV-2
detection assays

| | | Exam- |
|---|---|---|
| | | ple |
| | | of |
| Name | Sequences | Assay |
| RPA30_ | GCGCTAATACGACTCACTATAGGGAATTACAAT | FIG. |
| 201 | ACAAATAGAGGAGACGGACAATGTACACCATCG | 2D |
| 9_ | TCGAGCAATATGAAAGAGCTGAGGCTCGGCACT | |
| nCoV_ | CAACCAACCTGGCGGCAGCGCAAAAGGAGACAG | |
| C + | GTACGTTAATAGTTAATAGCGTA | |
| G_E_ | (SEQ ID NO: 1) | |
| fwd | | |
| RPA30_ | TTATCAGCCACCTCCTGTAATCCCAGCGGCATT | |
| 201 | GACATACTCGTGCAGAACCATGTGATCGCGCGA | |
| 9_ | TCCACCGCCACTACCTCCGCCGTCACTCAGGTT | |
| nCoV_ | AACAATATTGCAGCAGTACGCACACAA | |
| C + | (SEQ ID NO: 2) | |
| G_E_ | | |
| rev | | |
| RPA30_ | GCGCTAATACGACTCACTATAGGGAATTACAAT | FIG. |
| 201 | ACAAATAGAGGAGACGGACAATGTACACCATCG | 2E |
| 9_ | TCGAGCAATATGAAAGAGCTGAGGCTCGGCACT | |
| nCoV_ | CAACCAACCTGGCGGCAGCGCAAAAGCAGGAAC | |
| C + | TAATCAGACAAGGAACTGATTAC | |
| G_N_ | (SEQ ID NO: 3) | |
| fwd | | |
| RPA30_ | TTATCAGCCACCTCCTGTAATCCCAGCGGCATT | |
| 201 | GACATACTCGTGCAGAACCATGTGATCGCGCGA | |
| 9_ | TCCACCGCCACTACCTCCGCCGTCACTCAGCAA | |
| nCoV_ | CCACGTTCCCGAAGGTGTGACTTCCAT | |
| C + | (SEQ ID NO: 4) | |
| G_N_ | | |
| rev | | |
| RPA30_ | GCGCTAATACGACTCACTATAGGGAATTACAAT | FIG. |
| 201 | ACAAATAGAGGAGACGGACAATGTACACCATCG | 2F; |
| 9_ | TCGAGCAATATGAAAGAGCTGAGGCTCGGCACT | FIG. |
| nCoV_ | CAACCAACCTGGCGGCAGCGCAAAAGTCTGATA | 8 |
| C + | ATGGACCCCAAAATCAGCGAAAT | |
| G_CDC_ | (SEQ ID NO: 5) | |
| N1 | | |
| fwd | | |
| RPA30_ | TTATCAGCCACCTCCTGTAATCCCAGCGGCATT | |
| 201 | GACATACTCGTGCAGAACCATGTGATCGCGCGA | |
| 9_ | TCCACCGCCACTACCTCCGCCGTCACTCAGTCC | |
| nCoV_ | ATTCTGGTTACTGCCAGTTGAATCTGA | |
| C + | (SEQ ID NO: 6) | |
| G_CDC_ | | |
| N1 | | |
| rev | | |
| RPA30_ | GCGCTAATACGACTCACTATAGGGAATTACAAT | FIG. |
| 201 | ACAAATAGAGGAGACGGACAATGTACACCATCG | 2G |
| 9_ | TCGAGCAATATGAAAGAGCTGAGGCTCGGCACT | |
| nCoV_ | CAACCAACCTGGCGGCAGCGCAAAAGGTTCAAC | |
| C + | AATGGGGTTTTACAGGTAACCTA | |
| G_ | (SEQ ID NO: 7) | |
| ORF1b_ | | |
| fwd | | |
| RPA30_ | TTATCAGCCACCTCCTGTAATCCCAGCGGCATT | |
| 201 | GACATACTCGTGCAGAACCATGTGATCGCGCGA | |
| 9_ | TCCACCGCCACTACCTCCGCCGTCACTCAGAGT | |
| nCoV_ | CCAGTCAACACGCTTAACAAAGCACTC | |
| C + | (SEQ ID NO: 8) | |
| G_ | | |
| ORF1b_ | | |
| rev | | |
| RPA_ | TTTCTAATACGACTCACTATAGGGATCAGCAGC | FIG. |
| SARS | ATAAACAGAGGAGACCAGACATGTATACCATCG | 6B |
| 2_N_ | TTGAACAATACGAACGCGCAGAAGCAAGACACT | |
| | CCACCAACCTCGCCGCCGCCCAAAAATTGTTGG | |
| frame1_ | CCTTTACCAGACATTTTGCTCTCA | |
| cherry2_ | (SEQ ID NO: 9) | |
| fwd_ | | |
| primer4 | | |
| RPA_ | TTATTATCCTCCTCCCGTTATTCCCGCCGCGTT | |
| SARS | TACGTATTCATGCAGTACCATATGATCCCTCGA | |
| 2_N_ | GCCACCTCCTGATCCTCCTCCGTCGCTTAAATA | |
| frame1_ | GGCAGCAGTAGGGGAACTTCTCCTGCTAGAA | |
| GFP11_ | (SEQ ID NO: 10) | |
| rev | | |
| primer4 | | |
| RPA_ | CAACTAATACGACTCACTATAGGGATAAACAGA | FIG. |
| SARS | AGCAACAGAGGAGACCAAGGATGTATACAATCG | 6C |
| 2_N_ | TCGAACAATATGAAAGAGCCGAAGCCCGCCACA | |
| frame2 | GCACGAACTTAGCCGCCGCCCAAAAAAGCATTG | |
| A_ | TTAGCAGGATTGCGGGTGCCAATGTGA | |
| cherry2_ | (SEQ ID NO: 11) | |
| fwd_ | | |
| primer1 | | |
| RPA_ | TTATTAGCCACCGCCGGTTATGCCCGCTGCATT | |
| SARS | TACATATTCGTGCAGCACCATGTGATCTCGGGA | |
| 2_N_ | CCCGCCGCCTGAACCTCCTCCATCACTTAAGAA | |
| frame2 | GACGGCATCATATGGGTTGCAACTGAGGGA | |
| A_ | (SEQ ID NO: 12) | |
| GFP11_ | | |
| rev_ | | |
| primer1 | | |
| RPA_ | AAACTAATACGACTCACTATAGGGACAAGAACA | FIG. |
| SARS | ATAAGCAGAGGAGATGGACAATGTATACTATCG | 6D |
| 2_N_ | TCGAACAATACGAACGCGCCGAAGCACGCCATT | |
| frame2 | CCACCAACTTAGCGGCGGCACAGAAACTTGTTA | |
| B_ | GCAGGATTGCGGGTGCCAATGTGA | |
| cherry2_ | (SEQ ID NO: 13) | |
| fwd_ | | |
| primer3 | | |
| RPA_ | TTATTATCCGCCGCCGGTGATGCCCGCCGCGTT | |
| SARS | AACGTATTCGTGTAGTACCATGTGATCTCTGCT | |
| 2_N_ | TCCGCCTCCCGAGCCTCCGCCGTCAGATAAAAC | |
| frame2 | AAAGACGGCATCATATGGGTTGCAACTGAGGGA | |
| B_ | (SEQ ID NO: 14) | |
| GFP11_ | | |
| rev_ | | |
| primer3 | | |
| RPA_ | GAACTAATACGACTCACTATAGGGCACAGAAAC | FIG. |
| SARS | AGGAACAGAGGAGATAGAACATGTATACGATCG | 6E |
| 2_N_ | TCGAACAATATGAAAGAGCAGAAGCAAGACACA | |
| frame4_ | GCACAAACCTAGCAGCGGCCCAGAAAAAGCAGT | |
| cherry2_ | ATTATTGGGTAAACCTTGGGGCCGA | |
| fwd_ | (SEQ ID NO: 15) | |
| primer1 | | |
| RPA_ | TTATTAACCTCCACCCGTAATTCCCGCTGCGTT | |
| SARS | TACATATTCATGCAGCACCATATGATCCCTGCT | |
| 2_N_ | GCCTCCTCCGCTTCCGCCTCCGTCGCTTAGTGT | |
| frame4_ | TTGGTGGACCCTCAGATTCAACTGGCAGTAACC | |
| GFP11_ | A | |
| rev_ | (SEQ ID NO: 16) | |
| primer1 | | |
| RPA30_ | TTATCAGCCACCTCCCTCTTCGCTATTACGCCA | |
| 201 | GCTGGCGAAAGGGGGATGTGCTGCAAGGCGATT | |
| 9_ | AAGTTGGGTAACGCCAGGGTTTTCCCAGTCACG | |
| nCoV_ | ACGTTGAAGCACTACAGCAAGTGAATCCGTAAT | |
| alp | CATGGAACCGCCCCCGTCACTCAGCAACCACGT | |
| ha39AA_ | TCCCGAAGGTGTGACTTCCAT | |
| N_ | (SEQ ID NO: 17) | |
| rev | | |
| RPA30_ | TTATCACCCCCCACCTTCCTCACTGTTACGCCA | |
| 201 | GGAGGCAAATGGTGGATGAGCCGCCAGACGGTT | |
| 9_ | TAATTGAGTAACACCTGGATTTTCCCAATCACG | |
| nCoV_ | GCGCTGAAGCACTACAGCAAGTGAATCCGTAAT | |
| alp | CATGGAACCGCCCCCGGAGTCGGACAGCAACCA | |
| ha39AA2_ | CGTTCCCGAAGGTGTGACTTCCAT | |
| N_ | (SEQ ID NO: 18) | |
| rev | | |
| 2019_ | GCGCTAATACGACTCACTATAGGGAATTACAAT | |
| CoV_ | ACAAATAGAGGAGACGGACAATGTTCCATGCCA | |
| CDC_ | ATGCGCGACATTCCGAAGAA | |
| N2_S | (SEQ ID NO: 19) | |
| NP_F | | |
| 2019_ | TTATCAGCCACCTCCTGTAATCCCAGCGGCATT | |
| CoV_ | GACATACTCGTGCAGAACCATGTGATCGCGCGA | |
| CDC_ | TCCACCGCCACTACCTCCGCCGTCACTCAGCAA | |
| N2_S | GGAACTGATTACAAACATTGGCCGCAAA | |
| NP_R | (SEQ ID NO: 20) | |

TABLE 2
Primer sequences used in Example 2

| | | Exam- |
|---|---|---|
| | | ple |
| | | of |
| Name | Sequences | Assay |
| RPA_ | GCGCTAATACGACTCACTATAGGGAATTACAAT | FIG. |
| 68_ | ACAAATAGAGGAGACGGACAATGGTTGAAGAAG | 9 |
| 69F | TACAAAATGTCATTAATGCTATG | |
| | (SEQ ID NO: 21) | |
| lin_ | TTAGGTTGAGTGCCGAGCCTCAGCTCTTTCATA | |
| chry2_ | TTGCTCGACGATGGTGTAGCCCCCGGATCCCCC | |
| Term_ | GGTTGAGTGCCGAGCCTCAGCTCTTTCATATTG | |
| RPA_ | CTCGACGATGGTGTACGATCCACCGCCACTACC | |
| 68__ | TCCGCCAAGTTTCAGCATGCAAAATTTGCAAAA | |
| 69R | TATGTG | |
| | (SEQ ID NO: 22) | |
| RPA_ | GCGCTAATACGACTCACTATAGGGAATTACAAT | FIG. |
| 2475F | ACAAATAGAGGAGACGGACAATGTTTGAAAACC | 9 |
| | CCAAGGGACTAATTCATGGTTGT | |
| | (SEQ ID NO: 23) | |
| lin_ | TTAGGTTGAGTGCCGAGCCTCAGCTCTTTCATA | |
| chry2_ | TTGCTCGACGATGGTGTAGCCCCCGGATCCCCC | |
| Term_ | GGTTGAGTGCCGAGCCTCAGCTCTTTCATATTG | |
| RPA_ | CTCGACGATGGTGTACGATCCACCGCCACTACC | |
| 247 | TCCGCCCATTTCTATGCTTGTTTCCCGACTGTG | |
| 5R | GTTAAC | |
| | (SEQ ID NO: 24) | |
| RPA_ | GCGCTAATACGACTCACTATAGGGAATTACAAT | FIG. |
| 3029_ | ACAAATAGAGGAGACGGACAATGTCATTTGTTA | 9 |
| 3030F | AAACTAAATGTAAGAAAAATCTG | |
| | (SEQ ID NO: 25) | |
| lin_ | TTAGGTTGAGTGCCGAGCCTCAGCTCTTTCATA | |
| chry2_ | TTGCTCGACGATGGTGTAGCCCCCGGATCCCCC | |
| Term_ | GGTTGAGTGCCGAGCCTCAGCTCTTTCATATTG | |
| RPA_ | CTCGACGATGGTGTACGATCCACCGCCACTACC | |
| 3029_ | TCCGCCTTCTCTAATGTTATTACGGCTAATTGT | |
| 3030R | GCTCAC | |
| | (SEQ ID NO: 26) | |
| RPA_ | GCGCTAATACGACTCACTATAGGGAATTACAAT | FIG. |
| 4936_ | ACAAATAGAGGAGACGGACAATGACATCAAAAA | 9 |
| 4939F | GTATCTTTTTGAAAGTTAAAGTA | |
| | (SEQ ID NO: 27) | |
| lin_ | TTAGGTTGAGTGCCGAGCCTCAGCTCTTTCATA | |
| chry2_ | TTGCTCGACGATGGTGTAGCCCCCGGATCCCCC | |
| Term_ | GGTTGAGTGCCGAGCCTCAGCTCTTTCATATTG | |
| RPA_ | CTCGACGATGGTGTACGATCCACCGCCACTACC | |
| 4936_ | TCCGCCGACTGAATAAGGGGACTGATTTGTGTA | |
| 4939R | ACAAGT | |
| | (SEQ ID NO: 28) | |
| CFTR_ | TTTCTAATACGACTCACTATAGGGAATTACAAT | FIG. |
| W128 | ACAAATAGAGGAGACGGACAATGAGACTACTGA | 10 |
| 2X_ | ACACTGAAGGAGAAATCCAGAT | |
| F_1 | (SEQ ID NO: 29) | |
| CFTR_ | TCACGTGATGCCAGCGGCATTCACATACTCGTG | |
| W128 | CAGTACCATGTGGTCACGAGTCCTTTTGCTCAC | |
| 2X_ | CTGTGGTATCACTCCAA | |
| RevGFP_ | (SEQ ID NO: 30) | |
| 1 | | |
| CFTR_ | TTTCTAATACGACTCACTATAGGGAATTACAAT | FIG. |
| W128 | ACAAATAGAGGAGACGGACAATGATCCAGATCG | 10 |
| 2X_F_2 | ATGGTGTGTCTTGGGATTCAATA | |
| | (SEQ ID NO: 31) | |
| CFTR_ | TCACGTGATGCCAGCGGCATTCACATACTCGTG | |
| W128 | CAGTACCATGTGGTCACGAGTCCTTTTGCTCAC | |
| 2X_ | CTGTGGTATCACTCCAA | |
| RevGFP_ | (SEQ ID NO: 32) | |
| 1 | | |
| THMD2_ | TTTCTAATACGACTCACTATAGGGAATTACAAT | FIG. |
| F_4 | ACAAATAGAGGAGACGGACAATGGCTTTCCTTT | 11 |
| | TCTCACTTTTCCTACCAATGC | |
| | (SEQ ID NO: 33) | |
| THMD2_ | TCACGTGATGCCAGCGGCATTCACATACTCGTG | |
| Rev | CAGTACCATGTGGTCACGTGGATTCACGCTTGA | |
| GFP_4 | TGACTTCTTTATTTCTCT | |
| | (SEQ ID NO: 34) | |
| 5266dupC_ | TTTCTAATACGACTCACTATAGGGAATTACAAT | FIG. |
| F1 | ACAAATAGAGGAGACGGACAATGCAGCATGATT | 12 |
| | TTGAAGTCAGAGGAGAT | |
| | (SEQ ID NO: 35) | |
| 5266dupC_ | TCACGTGATGCCAGCGGCATTCACATACTCGTG | |
| 1 | CAGTACCATGTGGTCACGGATTTTTGTCAACTT | |
| GFP11_ | GAGGGAGGGAGCTTT | |
| Rev | (SEQ ID NO: 36) | |
| 1086_ | TTTCTAATACGACTCACTATAGGGAATTACAAT | FIG. |
| 1141_ | ACAAATAGAGGAGACGGACAATGATGCTGATCC | 12 |
| F1 | CCTGTGTGAGAGAAAAGAAT | |
| | (SEQ ID NO: 37) | |
| 1086_ | TCACGTGATGCCAGCGGCATTCACATACTCGTG | |
| 1141_ | CAGTACCATGTGGTCACGCTCCCCATCATGTGA | |
| GFP11_ | GTCATCAGAACCTAACA | |
| Rev | (SEQ ID NO: 38) | |
| 658_ | TTTCTAATACGACTCACTATAGGGAATTACAAT | FIG. |
| 659_ | ACAAATAGAGGAGACGGACAATGATTGTTAGCA | 12 |
| F_1 | ATTTCAACAGTCTAATCAATGTC | |
| | (SEQ ID NO: 39) | |
| 658_ | TCACGTGATGCCAGCGGCATTCACATACTCGTG | |
| 659_ | CAGTACCATGTGGTCACGTTTTATCTTACAGTC | |
| GFP11_ | AGAAATGAAGAAGCAT | |
| Rev | (SEQ ID NO: 40) | |
| 68_ | TTTCTAATACGACTCACTATAGGGAATTACAAT | FIG. |
| 69_ | ACAAATAGAGGAGACGGACAATGGAACAGAAAG | 12 |
| F_4 | AAATGGATTTATCTGCTCTTCG | |
| | (SEQ ID NO: 41) | |
| 68_ | TCACGTGATGCCAGCGGCATTCACATACTCGTG | |
| 69_ | CAGTACCATGTGGTCACGATCATAGGAATCCCA | |
| 4GFP | AATTAATACACTCTTGT | |
| 11_ | (SEQ ID NO: 42) | |
| Rev | | |
| Dual_ | TTTCTAATACGACTCACTATAGGGAATTACAAT | FIG. |
| RPA_6 | ACAAATAGAGGAGACGGACAATGTACACCATCG | 13 |
| 58_ | TCGAGCAATATGAAAGAGCTGAGGCTCGGCACT | |
| 659delF_ | CAACCTCTTTAGCTACACCACCCACCCTTAGTT | |
| chery2 | CT | |
| | (SEQ ID NO: 43) | |
| Dual_ | TCACGTGATGCCAGCGGCATTCACATACTCGTG | |
| RPA_6 | CAGTACCATGTGGTCACGTCTATCATTTTTCTT | |
| 58_ | CAGACTTTCATCATG | |
| 659delR | (SEQ ID NO: 44) | |
| Dual_ | TTTCTAATACGACTCACTATAGGGAATTACAAT | FIG. |
| RPA_6 | ACAAATAGAGGAGACGGACAATGTACACCATCG | 13 |
| 8_69F_ | TCGAGCAATATGAAAGAGCTGAGGCTCGGCACT | |
| chery2 | CAACCGTTGAAGAAGTACAAAATGTCATTAATG | |
| | CTATG | |
| | (SEQ ID NO: 45) | |
| Dual_ | TCACGTGATGCCAGCGGCATTCACATACTCGTG | |
| RPA_6 | CAGTACCATGTGGTCACGAAGTTTCAGCATGCA | |
| 8_69R | AAATTTGCAAAATATGTG | |
| | (SEQ ID NO: 46) | |
| Dual_ | TTTCTAATACGACTCACTATAGGGAATTACAAT | FIG. |
| RPA_5 | ACAAATAGAGGAGACGGACAATGTACACCATCG | 13 |
| 266F_ | TCGAGCAATATGAAAGAGCTGAGGCTCGGCACT | |
| chery2 | CAACCAATGAGCATGATTTTGAAGTCAGAGGAG | |
| | AT | |
| | (SEQ ID NO: 47) | |
| Dual_ | TCACGTGATGCCAGCGGCATTCACATACTCGTG | |
| RPA_5 | CAGTACCATGTGGTCACGATAGCAACAGATTTC | |
| 266R | TAGCCCCCTGAAGAT | |
| | (SEQ ID NO: 48) | |
| Dual_ | TTTCTAATACGACTCACTATAGGGAATTACAAT | FIG. |
| RPA_1 | ACAAATAGAGGAGACGGACAATGTACACCATCG | 14 |
| 086_ | TCGAGCAATATGAAAGAGCTGAGGCTCGGCACT | |
| 1141F_ | CAACCGTAGATCTGAATGCTGATCCCCTGTGTG | |
| chery2 | AG | |
| | (SEQ ID NO: 49) | |
| Dual_ | TCACGTGATGCCAGCGGCATTCACATACTCGTG | |
| RPA_1 | CAGTACCATGTGGTCACGCTCCCCATCATGTGA | |
| 086_ | GTCATCAGAACCTAA | |
| 1141R | (SEQ ID NO: 50) | |
| Dual_ | TTTCTAATACGACTCACTATAGGGAATTACAAT | FIG. |
| RPA_2 | ACAAATAGAGGAGACGGACAATGTACACCATCG | 14 |
| 475F_ | TCGAGCAATATGAAAGAGCTGAGGCTCGGCACT | |
| chery2 | CAACCTTTGAAAACCCCAAGGGACTAATTCATG | |
| | GTTGT | |
| | (SEQ ID NO: 51) | |
| Dual_ | TCACGTGATGCCAGCGGCATTCACATACTCGTG | |
| RPA_2 | CAGTACCATGTGGTCACGCATTTCTATGCTTGT | |
| 475R | TTCCCGACTGTGGTTAAC | |
| | (SEQ ID NO: 52) | |
| Dual_ | TTTCTAATACGACTCACTATAGGGAATTACAAT | FIG. |
| RPA_4 | ACAAATAGAGGAGACGGACAATGTACACCATCG | 14 |
| 936_ | TCGAGCAATATGAAAGAGCTGAGGCTCGGCACT | |
| 4939F_ | CAACCACATCAAAAAGTATCTTTTTGAAAGTTA | |
| chery2 | AAGTA | |
| | (SEQ ID NO: 53) | |
| Dual_ | TCACGTGATGCCAGCGGCATTCACATACTCGTG | |
| RPA_4 | CAGTACCATGTGGTCACGGACTGAATAAGGGGA | |
| 936_ | CTGATTTGTGTAACAAGT | |
| 4939R | (SEQ ID NO: 54) | |
| 2475delC | TTTCTAATACGACTCACTATAGGGAATTACAAT | FIG. |
| _F_3 | ACAAATAGAGGAGACGGACAATGTACACCATCG | 15 |
| | TCGAGCAATATGAAAGAGCTGAGGCTCGGCACT | |
| | CAACCTGTGCAGCATTTGAAAACCCCAAGGGAC | |
| | TA | |
| | (SEQ ID NO: 55) | |
| 2475de1C | TCACGTGATGCCAGCGGCATTCACATACTCGTG | |
| _F_3 | CAGTACCATGTGGTCACGCATTTCTATGCTTGT | |
| GFP11_ | TTCCCGACTGTGGTTAAC | |
| Rev | (SEQ ID NO: 56) | |
| 3029_ | TTTCTAATACGACTCACTATAGGGAATTACAAT | FIG. |
| 3030de | ACAAATAGAGGAGACGGACAATGTACACCATCG | 15 |
| 1_1 | TCGAGCAATATGAAAGAGCTGAGGCTCGGCACT | |
| | CAACCAGGAAAACTTTGAGGAACATTCAATGTC | |
| | AC | |
| | (SEQ ID NO: 57) | |
| 3029_ | TCACGTGATGCCAGCGGCATTCACATACTCGTG | |
| 3030de | CAGTACCATGTGGTCACGAACATTTTCTCTAAT | |
| 1_1 | GTTATTACGGCTAATTGTGC | |
| GFP11_ | (SEQ ID NO: 58) | |
| Rev | | |
| 2679_ | TTTCTAATACGACTCACTATAGGGAATTACAAT | FIG. |
| 2682_1 | ACAAATAGAGGAGACGGACAATGTACACCATCG | 15 |
| | TCGAGCAATATGAAAGAGCTGAGGCTCGGCACT | |
| | CAACCTTTTCAAATCCAGGAAATGCAGAAGAGG | |
| | AAT | |
| | (SEQ ID NO: 59) | |
| 2679_ | TCACGTGATGCCAGCGGCATTCACATACTCGTG | |
| 2682_1 | CAGTACCATGTGGTCACGCTGTACAGGCTTGAT | |
| GFP11_ | ATTAGACTCATTCTTT | |
| Rev | (SEQ ID NO: 60) | |

We claim:

1. A method of detecting a target nucleic acid in a sample, the method comprising the steps of:
(a) amplifying a nucleic acid obtained from a sample of a subject using a forward peptide primer and a reverse peptide primer,
wherein the forward peptide primer comprises, from 5′ to 3′, a nucleic acid sequence encoding a ribosome binding site (RBS), a start codon, and a nucleic acid sequence of a first portion of the target nucleic acid sequence, wherein the first portion is optionally a protein coding sequence in-frame with the start codon; and
wherein the reverse peptide primer comprises, from 3′ to 5′, a nucleic acid sequence complementary to a second portion of the target nucleic acid sequence, a reverse complement of a sequence encoding a peptide reporter, and a reverse complement of a stop codon in-frame with the reverse complement of the sequence encoding the peptide reporter;
(b) translating the nucleic acid into translation products using a cell-free translation reaction; and
(c) detecting a translated peptide reporter, if present, among the translation products, wherein the presence of the peptide reporter indicates the presence of the target nucleic acid in the sample.

2. The method of claim 1, wherein step (c) comprises contacting the translation products with an agent which binds to the peptide reporter; optionally wherein the agent is a detectable agent.

3. The method of claim 2, wherein the agent is a detectable agent comprising an antibody or antibody fragment which binds the peptide reporter and wherein the detecting step (c) comprises detecting the peptide reporter by detecting the detectable agent.

4. The method of claim 1, wherein the translating step (b) comprises a cell-free translation reaction comprising one or more reagents that binds to the peptide reporter to form at least one detectable agent; and wherein the detecting step (c) comprises detecting the peptide reporter by detecting the at least one detectable agent.

5. The method of any one of claims 1-4, wherein the forward peptide primer further comprises, 5′ to the RBS, a transcriptional promoter; and wherein the cell-free translation reaction is a transcription-translation reaction wherein transcription of the amplified nucleic acid of the amplifying step (a) into a peptide reporter mRNA and translation of the peptide reporter mRNA into a translated peptide reporter is detected in the detecting step (c).

6. The method of claim 5, wherein the transcriptional promoter comprises a T7 RNA polymerase promoter.

7. The method of any one of claims 1-6, wherein the RBS is prokaryotic, the start codon is prokaryotic, the stop codon is prokaryotic, or any combination thereof.

8. The method of any one of claims 1-7, wherein amplifying comprises a polymerase chain reaction (PCR).

9. The method of any one of claims 1-7, wherein amplifying is isothermal.

10. The method claim 9, wherein amplifying comprises a recombinase polymerase amplification (RPA) or reverse-transcription recombinase polymerase amplification assay (RT-RPA).

11. The method of any one of claims 2-10, wherein the detectable agent is fluorescent, luminescent, or capable of catalyzing a colorimetric reaction.

12. The method of any one of claims 1-11, wherein the peptide reporter comprises a GFP11 peptide, lacZ-α peptide, sfCherry11 peptide, or sfCherry₂11 peptide.

13. The method of claim 12, wherein the peptide reporter comprises a lacZ-α peptide, wherein step (c) comprises contacting the translation products with a detectable agent comprising LacZ-ω protein which binds to the peptide reporter, and wherein the detectable agent is detected by a colorimetric reaction comprising chlorophenol red-β-D-galactopyranoside.

14. The method of claim 12, wherein the peptide reporter comprises:
(i) a GFP11 peptide and wherein step (c) comprises contacting the translation products with an agent comprising GFP1-10 protein which binds to the peptide reporter to form a fluorescent detectable agent;
(ii) a sfCherry11 peptide and wherein step (c) comprises contacting the translation products with an agent comprising sfCherry1-10 protein which binds to the peptide reporter to form a fluorescent detectable agent; or
(iii) a sfCherry₂11 peptide and wherein step (c) comprises contacting the translation products with an agent comprising sfCherry₂1-10 protein which binds to the peptide reporter to form a fluorescent detectable agent.

15. The method of any one of claims 1-14, wherein the detecting step can detect the peptide reporter when the target nucleic acid is at a concentration as low as 50 aM in the sample.

16. The method of any one of claims 1-15, wherein the method is performed in less than 6 hours, in less than 3 hours, in less than 2 hours, in less than 1 hour, or in less than 30 minutes.

17. The method of any one of claims 1-16, wherein the sample is a biological sample obtained from a subject.

18. The method of claim 17, wherein the subject is human.

19. The method of any one of claims 1-18, wherein the forward peptide primer further comprises a nucleic acid sequence encoding a second peptide reporter positioned 3′ of the start codon, 5′ of the nucleic acid sequence complementary to the first portion of the target nucleic acid sequence, and in-frame with the start codon; and wherein the detecting step (c) comprises detecting the translated second peptide reporter and optionally detecting the peptide reporter.

20. The method of claim 19, further comprising determining if the target nucleic acid comprises a frame-altering mutation or nonsense mutation, wherein when the second peptide reporter can be detected and the peptide reporter is not detected the target nucleic acid sequence comprises the frame-altering mutation or nonsense mutation.

21. The method of any one of claims 1-18, further comprising determining if the target nucleic acid comprises a single nucleotide polymorphism (SNP) or single nucleotide variant (SNV), wherein the first peptide primer comprises a nucleotide corresponding to the SNP or SNV within the nucleic acid sequence of the first portion of the target nucleic acid sequence or the second peptide primer comprises a nucleotide corresponding to the SNP or SNV within the nucleic acid sequence complementary to the second portion of the target nucleic acid sequence.

22. The method of claim 21, wherein the nucleotide corresponding to the SNP or SNV is positioned within the first five nucleotides of the most 3′ nucleotides of (a) the nucleic acid sequence of the first portion of the target nucleic acid or (b) the nucleic acid sequence complementary to the second portion of the target nucleic acid sequence.

23. A composition comprising:
(a) a forward primer comprising, from 5′ to 3′, a nucleic acid sequence encoding a ribosome binding site (RBS), a start codon, and a nucleic acid sequence of a first portion of a target nucleic acid sequence; and
(b) a reverse primer comprising, from 5′ to 3′, a reverse complement of a stop codon in-frame with the reverse complement of the sequence encoding a first peptide reporter in frame with a nucleic acid sequence complementary to a second portion of the target nucleic acid sequence.

24. The composition of claim 23, comprising a forward primer comprising:
(a) a transcriptional promoter 5′ of the nucleic acid sequence encoding a ribosome binding site (RBS);
(b) a nucleic acid sequence encoding a ribosome binding site (RBS), a start codon, a nucleic acid sequence encoding a second peptide reporter that is positioned 3′ of the start codon, 5′ of the nucleic acid sequence of the first portion of the target nucleic acid sequence, and in-frame with the start codon; or
(c) a transcriptional promoter 5′ of the nucleic acid sequence encoding a ribosome binding site (RBS), and a nucleic acid sequence encoding a second peptide reporter that is positioned 3′ of the start codon, 5′ of the nucleic acid sequence of the first portion of the target nucleic acid sequence, and in-frame with the start codon.

25. A composition comprising a forward primer and a reverse primer comprising a nucleotide sequence having at least 90% identity respectively to SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, and SEQ ID NO: 59 and SEQ ID NO: 60.

26. A composition comprising a forward primer and a reverse primer respectively comprising the nucleotide sequence selected from SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, and SEQ ID NO: 59 and SEQ ID NO: 60.

27. A kit comprising:
(a) a composition according to any one of claims 23-26,
(b) a reagent or device;
optionally wherein the reagent comprises:
an agent capable of binding the first or second peptide reporter,
an agent capable of binding the first or second peptide reporter to form a detectable agent,
a detectable agent which binds the first or second peptide reporter;
reagents for performing an amplification;
reagents for performing a cell-free translation reaction;
reagents for performing a cell-free transcription-translation reaction; or a paper test article; and
optionally wherein the device is:
portable,
adapted for performing a nucleic acid amplification,
adapted for performing a cell-free translation reaction,
adapted for performing a cell-free transcription-translation reaction, and/or
adapted for detecting the first or second peptide reporter;
and/or optionally wherein the device comprises a light or color sensor.
